(19) United States
(12) Patent Application Publication
(10) Pub. No.: US 2012/0309811 A1
(43) Pub. Date: Dec. 6, 2012

(54) P-GLYCOPROTEIN-SPECIFIC NON-COMPETITIVE PEPTIDE AND PEPTIDOMIMETIC MODULATORS

(75) Inventors: Ophelie Arnaud, Saint Martin Sur La Chambre (FR); Ali Koubeissi, Khaldel (LB); Laurent Ettouati, Lyon (FR); Joelle Paris, Lyon (FR); Attilio Di Peetro, Manziat (FR); Pierre Falson, Annonay (FR)

(73) Assignees: UNIVERSITE CLAUDE BERNARD - LYON 1, Villeurbanne Cedex (FR); CENTRE NATIONAL DE LA RECHERCHE SCIENTIFIQUE - CNRS -, Paris Cedex 16 (FR)

(21) Appl. No.: 13/500,268

(22) PCT Filed: Oct. 5, 2010
(86) PCT No.: PCT/FR2010/052095

§ 371 Date: Aug. 23, 2012

(30) Foreign Application Priority Data
Oct. 6, 2009 (FR) 0956954

(51) Int. Cl. A61K 31/40; A61P 31/00; C12N 5/071; A61P 35/00

(52) U.S. Cl. 514/423; 514/425; 435/375

(57) ABSTRACT

The invention relates to a compound having structure (I), which acts as a non-competitive specific inhibitor of P-glycoprotein (P-gp for “Pleiotropic glycoprotein”) and which can be used as a drug, particularly for improving the effectiveness of chemotherapy treatments. The invention is therefore suitable for use in the medical field, particularly for the chemotherapeutic treatment of cancers or infections.

### TECHNICAL FIELD

The present invention relates to a compound having structure (I), which acts as a non-competitive specific inhibitor of P-glycoprotein (P-gp for “Pleiotropic glycoprotein”), and which can be used as a medicament, in particular for improving the effectiveness of chemotherapy treatments.

The present invention is therefore suitable for use in particular in the medical field, in particular in the context of chemotherapy treatments for cancers or for infections.

In the description below, the references between square brackets ([ ]) refer back to the list of references provided after the examples.

### STATE OF THE ART

Chemotherapy treatments are currently used to combat cancers and infections of viral, bacterial, fungal or parasitic origin, by destroying the target cells of the treatment represented by the tumor cells or the cells infected with the infectious agent (virus, bacterium, fungus, parasite, etc.). Their effectiveness is often limited by the resistance that the target cells develop against the chemotherapeutic agents administered in the context of the treatment. Among acquired resistance phenomena, multidrug resistance (also called MDR) results in a decrease in the intracellular concentration of the chemotherapeutic agent. The target cells become resistant not only to the chemotherapeutic agents administered, but also to a large number of structurally unrelated molecules.

This decrease in the intracellular concentration of the chemotherapeutic agent is caused by the massive expression of transport proteins of “ATP-binding cassette” (ABC) type, which expel the chemotherapeutic agent out of the target cells. Three of these proteins, also called efflux pumps, have been identified in humans:
P-glycoprotein (P-gp) (Gottesman M. M., Ling V. FEBS Lett., 2006, 580(4), 998-1009, [1]), also called MDR1 or ABCB1, discovered by Juliano and Ling in 1976 (Juliano, R. L. & Ling, V. A surface glycoprotein modulating drug permeability in Chinese hamster ovary cell mutants. Biochim Biophys Acta 455, 152-162, doi:0005-2736(76)90160-7 [pii] (1976), [2]);
MRP1 (Multidrug Resistance Protein 1), also called ABCC1, discovered by Cole and Deeley in lung cancers (Cole, S. P. et al. Overexpression of a transporter gene in a multidrug-resistant human lung cancer cell line. Science 258, 105D-1654(1992), [3]);
Breast Cancer Resistance Protein (BCRP) (Doyle L. A., Yang W., Abruzzo L. V., Krogmann T., Gao Y., Rishi A. K., Ross D. D. Proc. Natl. Acad. Sci. USA, 1998, 95(26), 15665-15670, [4]) also called ABCG2 or MXR (mitoxantrone resistance), discovered more recently by firstly Ross et al_{—} (Ross, D. D. et al. Atypical multidrug resistance: breast cancer resistance protein messenger RNA expression in mitoxantrone-selected cell lines. J Natl Cancer Inst 91, 429-433 (1999), [5]) and, secondly, Bates et al. (J. Cell Sci., 2000, 113(Pt 11), 2011-2021, [6]).

These efflux proteins are embedded in the plasma membrane of cells via a transmembrane domain linked to a broad intracellular domain composed of two nucleotide-binding sites (Nucleotide-Binding Domain, NBD) and an extracellular domain. The three-dimensional structure of P-gp has recently been determined (Aller, S. G. et al. Structure of P-Glycoprotein Reveals a Molecular Basis for Poly-Specific Drug Binding. Science 323, 1718-1722, doi:10.1126/science.1168750 (2009), [7]) as close to that of the 2 prokaryotic efflux proteins, Sav1866 (Dawson, R. J. & Locher, K. P. Structure of a bacterial multidrug ABC transporter. Nature 443, 180-185 (2006), [8]) and MsbA (Ward, A., Reyes, C. L., Yu, J._{'} Roth, C. B. & Chang, G. Flexibility in the ABC transporter MsbA: Alternating access with a twist. Proceedings of the National Academy of Sciences 104, 19005-19010, doi:10.1073/pnas.0709388104 (2007), [9]). They allow the ATP-dependent efflux of various cytotoxic agents out of the cell.

p-gp (Ueda, K., Cardarelli, C., Gottesman, M. M. & Pastan, I. Expression of a Full-Length cDNA for the Human “MDR1” Gene Confers Resistance to Colchicine. Doxorubicin, and Vinblastine. Proceedings of the National Academy of Sciences 84, 3004-3008, doi:10.1073/pnas.84.9.3004 (1987), [10]) is the protein most widely implicated in drug resistance phenomena, reinforced in its action by MRP1 ([3]) and BCRP1 (Litman, T. et al. The multidrug-resistant phenotype associated with overexpression of the new ABC half-transporter, MXR (ABCG2). J Cell Sci 113 (Pt 11), 2011-2021 (2000), [11]).

P-gp has a physiological role that consists in protecting various organs; it participates in particular in the molecular mechanism allowing permeability of the blood-brain barrier (Juliano, R. L. & Ling, V. A surface glycoprotein modulating drug permeability in Chinese hamster ovary cell mutants. Biochim Biophys Acta 455, 152-162, doi:0005-2736(76)90160-7 [pii] (1976) [13]). Thus, several anticancer agents, such as doxorubicin and vinca alkaloids, are actively rejected out of the cells by P-gp ([5]). This makes it possible to explain the limited effectiveness of chemotherapy in the treatment of brain tumors. In the testicles, P-gp allows protection of the germ cells against xenobiotic compounds. The presence of P-gp in the placenta reflects its regulatory role on transfer of xenobiotics from the mother to the fetus ([8]). Many medicaments are substrates for P-gp (paclitaxel, daunorubicin or mitoxantrone, for example). This protein is also involved in the bioavailability of medicaments ([9]).

P-gp has recently been associated with the development of antiviral treatment resistance, by modulating the oral bioavailability of protease inhibitors and their penetration into the central nervous system (Kim, R. B. Drug transporters in HIV Therapy. Top HIV Med 11, 136-139 (2003), [15]). The transport spectrum of these pumps is very broad, which means that they are capable of bringing about the efflux of a large number of compounds, of varied nature, which are used for example for treating various pathological conditions, including cancer.

MRP1 is a polytopic transmembrane protein that transports conjugated organic anions such as cysteinyl leukotriene (LTC(4)). This transporter confers resistance to vincristine, to anthracyclines and to etoposide. MRP1 has a broad tissue distribution, including the basolateral membrane of epithelial cells of most tissues; it is expressed at a relatively high level in the lungs, the testicles and the kidneys. Overexpression of MRP1 has been observed in lung cancer, breast cancer, prostate cancer, cervical cancer, and leukemias treated with chemotherapy.

BCRP is present in the plasma membrane of cells. This protein was discovered in the placenta (Allikments R., Schriml L. M., Hutchinson A., Romano-Spica V., Dean M. Cancer Res., 1998, 58(23), 5337-5339, [16]) and then in breast cancer. The BCRP level in the placenta is 100 times higher than that found in the brain, prostate, small intestine, testicles, ovaries, colon and liver. BCRP is associated with multiresistance of certain cancer cells, but also performs various physiological functions. Indeed, it contributes to the absorption, distribution, metabolism and excretion of certain xenobiotics. Furthermore, BCRP is thought to participate significantly in the transport of natural substances such as plant polyphenols (Cooray H. C., Janvilisri T., Van Veen H. W., Hladky S. B., Barrand M. A. Biochem. Biophys. Res. Commun., 2004, 317(1), 269-275, [17]) and in protection of the fetus against certain toxic substances from maternal blood. BCRP reduces topotecan absorption (Jonker J. W., Smit J. W., Brinkhuis R. F., Maliepaard M., Beijnen J. H., Schellens J. H. J. Natl. Cancer Inst., 2000, 92, 1651-1656, [18]) at the intestinal level; this protein is therefore involved in the pharmacokinetics and bioavailability of medicaments.

These efflux proteins are therefore naturally expressed in normal tissues in order to expel toxins and waste produced by the cells (e.g., lung, kidney and liver). They are also produced at the level of the blood-brain barrier in order to prevent toxins from penetrating into the brain.

Several studies show the impact of the expression of these transporters on life expectancy and remission expectancy of patients treated by chemotherapy. Resistance to chemotherapy is in particular illustrated in FIG. 1, which is taken from the study published by Benderra et al. (Benderra Z. et al. Breast Cancer Resistance Protein and P-glycoprotein in 149 Adult Acute Myeloid Leukemias. Clin Cancer Res 10, 7896-7902, doi:10.1158/1078-0432.ccr-04-0795 (2004) [12]), which shows that life expectancy and percentage remission decrease when patients suffering from acute myeloid leukemia develop an overexpression of P-gp and/or of BCRP.

One of the potential strategies for reducing the impact of the expression of these transporters on chemotherapy treatment consists in coadministering the chemotherapeutic agent with an inhibitor of these efflux proteins. Several molecules have been developed for this purpose, and three generations of inhibitors are currently known. In this regard, tables 1a and 1b show the activity of MDR inhibitors on the various ABC transporters (Modok, S., Mellor, H. R. & Callaghan, R. Modulation of multidrug resistance efflux pump activity to overcome chemoresistance in cancer. Current Opinion in Pharmacology 6, 350-354 (2006), [19]). The symbols indicate either that the inhibitor is active (+) or that it is inactive (−) on a specified transporter, or that the effect is unknown (?).

TABLE 1a

Second generation of inhibitors

| Transporter | PSC833 (valspodar) | VX-710 (biricodar) |
|---|---|---|
| ABCB1 | + | + |
| ABCC1 | + | + |
| ABCG2 | − | + |

TABLE 1b

| | Third generation of inhibitors | | | | |
|---|---|---|---|---|---|
| | | | | | ONT- |
| | | | | | 093 |
| Trans- | GF120918 | R101933 | XR9576 | Ly335979 | (ontog- |
| porter | (elacridar) | (laniquidar) | (tariquidar) | (zosuquidar) | eny) |
| ABCB1 | + | + | + | + | + |
| ABCC1 | − | ? | − | − | − |
| ABCG2 | + | ? | + | − | ? |

Some inhibitors, such as tariquidar (Chemical Abstract Service number 206873-63-4) for example, have several targets. This is not necessarily an advantage since, ideally, a reversible agent, i.e. efflux protein inhibitor, should target only one efflux protein at a time in order to preserve the physiological functions of the others in healthy cells. Other agents, such as zosuquidar (Chemical Abstract Service number 167354-41-8), have recently been stopped in phase III because of their adverse effects.

Indeed, transport proteins have redundant efflux spectra, which slows down the development of modulators of these proteins since the modulator of a given transporter could impair the functioning of another transporter.

Furthermore, it is important to preserve the physiological functions of these transporters if they are not involved in a given resistance.

Hydrophobic dipeptides or tripeptides called reversins have P-gp activity-inhibiting properties, without any toxic effect at the effective doses used (Sarkadi B., Seprödi J., Csuka O., Magocsi M., Mezõ I., Palyi I., Teplán I., Vadász Z., Vincze, B. U.S. Pat. No. 6,297,216 B1, 2 Oct. 2001, [21], Seprödi J., Mezõ I., Vadász Zs., Szabó K., Sarkadi B., Teplán I. in peptides 1996, Proceedings of the Twenty-Fourth European Peptide Symposium: Sep. 8-13, 1996, Edinburgh, Scotland. Robert Ramage and Roger Epton (Eds), 1998 pp. 801-802 [22]). In particular, the most active reversins are reversins 121, 1092 and 213. These hydrophobic dipeptides have been studied by Sharom et al. (Sharom F. J., Yu X., Lu P., Liu R., Chu J. W. K., Szabó K., Muller M., Hose C. D., Monks A., Varadi A., Sepródi J., Sarkadi B. Biochem. Pharmacol., 1999, 58(4), 571-586 [23]) and have been found to have a high affinity for P-gp, with a submicromolar binding constant and an inhibition constant of about one micromolar.

However, even though the development of inhibitors of MDR-type ABC transporters is a major approach for combating cancer chemoresistance, this concept remains perfectable since it is based essentially on the search for compounds capable of competing with the substrate at the level of the transport site of these proteins.

The thesis document by A. Koubeissi (Thèse, Synthèse et étude biologique d'analogues di- et tripeptidiques de réversines susceptibles de moduler l'activité de deux protéines de transport, la glycoprotéine P et BCRP [Thesis, Synthesis and biological study of di- and tripeptide analogs of reversins capable of modulating the activity of two transport proteins, P-glycoprotein and BCRP] (2007), [24]) emphasizes that the major constraint of this approach is that the inhibitors thus selected can become substrates for other MDR transporters, capable of adapting their transport site, which is intrinsically polyspecific. In this context, this document describes the synthesis of inhibitors that are analogs of these reversins in order to specify the structural requirements necessary for activity with respect to P-gp and BCRP. However, this document does not describe these analogs as active or capable of application in the context of a chemotherapy treatment.

These examples demonstrate that there remains a real need to provide new compounds capable of being used in the context of a therapeutic treatment, which behave as specific inhibitors of an efflux pump and which do not exhibit significant inhibition of efflux pumps that are not involved in the resistance in question.

### DESCRIPTION OF THE INVENTION

The invention in fact makes it possible to overcome the drawbacks of the prior art and to meet these needs.

Indeed, the inventors provide a family of P-gp-modulating compounds or P-gp inhibitors which can be used as a medicament, or for preparing a medicament, that can be used in particular in chemotherapy treatments, or in pharmaceutical compositions that can be used in mammals, in particular in humans or animals.

Thus, the present invention relates to the use of a compound of formula (I), optionally in hydrated or solvated form, or a pharmaceutically acceptable salt thereof,

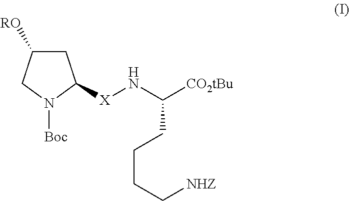

for preparing a medicament, wherein:
R represents a Bn, —COBn, —COcHex or —CH₂cHex group,
X represents —CH₂ or —CO,
Z represents a benzyloxycarbonyl group.

In the context of the invention, the following abbreviations are used: Boc for tert-butyloxycarbonyl, Bn for benzyl, cHex for cyclohexyl. Hyp for 4-hydroxyproline, Lys for lysine, Z for benzyloxycarbonyl, tBu for tert-butyl, O for ortho, Ser for serine, and Me for methyl.

In other words, the invention relates to a compound of formula (I) or a salt thereof as defined above, for use as a medicament.

It goes without saying that the stereoisomers, the mixtures and also the salts of the compound of formula (I) are part of the invention.

The compound of formula (I) as a medicament has the advantage of not inducing significant inhibition of MRP1 or of BCRP, thus preserving their respective physiological functions.

The compound of formula (I) as a medicament has the following advantages: a specificity for P-gp, an affinity for P-gp which is better than micromolar (i.e. less than or equal to 1 micromolar), for example up to 7 times better than that of reversin 121 for P-gp, a better level of inhibition than that of reversin 121, few or no adverse effects and a lack of competition with the substrate(s) transported by the pump.

When X represents —CH₂, R can be chosen from -Bn, —COBn, —COcHex or —CH₂cHex.

For example, X can represent CH₂ and R can represent Bn.

When X represents —CO, R can be chosen from -Bn, —COBn, —COcHex or —CH₂CHex.

For example, X can represent —CO and R can represent —COBn.

In another example, X can represent —CO and R can represent COcHex.

In another example, X can represent —CO and R can represent Bn.

In another example, X can represent —CO and R can represent CH₂cHex.

The compound can have one of the formulae chosen from formula (II), (III), (IV), (V) or (VI) hereinafter denoted respectively as “compound No. 6”, “compound No. 2”, “compound No. 3”, “compound No. 4” and “compound No. 5”:

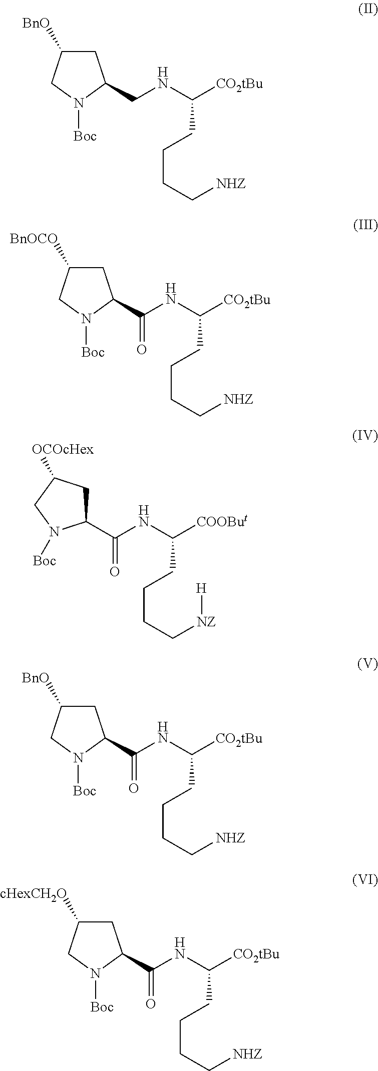

The compounds having the above formulae also comprise those in which one or more hydrogen, carbon or halogen, in particular fluorine or chorine, atoms have been replaced with their radioactive isotope, for example tritium or carbon 14.

All these compounds have the following properties: a lack of significant inhibition of MRP1 and of BCRP, a specificity with respect to P-gp, an affinity for P-gp that is better than micromolar (i.e. less than or equal to 1 micromolar), for example up to 7 times better than that of reversin 121 for P-gp, a better level of inhibition than that of reversin 121, few or no adverse effects and no competition.

The invention is taken to mean a composition comprising a single type of compound having a formula previously defined, or a mixture of at least two of these compounds.

When the compounds are in the form of a mixture, the proportions of each of the compounds will be determined by those skilled in the art, for example according to the mode of administration selected.

The mixture of these compounds has the following properties: a lack of significant inhibition of MRP1 and of BCRP, a specificity with respect to P-gp, an affinity for P-gp that is better than micromolar (i.e. less than or equal to 1 micromolar), for example up to 7 times better than that of reversin 121 for P-gp, a better level of inhibition than that of reversin 121, few or no adverse effects and no competition.

The compounds of formula previously defined can be in the form of pure isomers or in the form of a mixture of isomers.

The mixture of these isomers can be determined by those skilled in the art, for example according to the method of administration selected.

The mixture of these isomers has the advantage of not inducing significant inhibition of MRP1 and of BCRP.

The mixture of these isomers has the following advantages: a specificity for P-gp, an affinity for P-gp which is better than micromolar (i.e. less than or equal to 1 micromolar), for example up to 7 times better than that of reversin 121 for P-gp, a better level of inhibition than that of reversin 121, few or no adverse effects and no competition.

The salts of the compounds of formula previously defined comprise those with inorganic or organic acids, and the pharmaceutically acceptable salts.

As a suitable acid, mention may be made of oxalic acid or an optically active acid, for example a tartaric acid, a dibenzoyltartaric acid, a mandelic acid or a camphorsulfonic acid, and those which form physiologically acceptable salts, such as the hydrochloride, hydrobromide, sulfate, hydrogen sulfate, dihydrogen sulfate, maleate, fumarate, 2-naphthalenesulfate, para-toluenesulfonate, mesylate, besylate or isothionate.

The mixture of these isomers has the advantage of not inducing significant inhibition of MRP1 and of BCRP.

The mixture of these isomers has the following advantages: a specificity for P-gp, an affinity for P-gp which is better than micromolar (i.e. less than or equal to 1 micromolar), for example up to 7 times better than that of reversin 121 for P-gp, a better level of inhibition than that of reversin 121, few or no adverse effects and no competition.

As compounds in hydrated form, mention may be made, by way of example, of the hemihydrates, and monohydrates.

The compounds of the invention can be prepared by any process known to those skilled in the art. The processes described in the document A. Koubeissi ([24]) can, for example, be used. The synthesis of the compounds of the invention can be carried out by peptide coupling of the protected derivative of trans-4-hydroxy-L-proline with commercial H-Lys(Z)-OtBu.HCl by the mixed anhydride method (Lai, M. Y. H. et al. Synthesis and pharmacological evaluation of glycine-modified analogs of the neuroprotective agent glycyl-l-prolyl-l-glutamic acid (GPE). Bioorganic & Medicinal Chemistry 13, 533-548 (2005), [25]). The aminomethyl analog compound No. 6 can be obtained by reductive amination under conventional conditions (Martinez, J. et al. Synthesis and biological activities of some pseudo-peptide analogs of tetragastrin: the importance of the peptide backbone. Journal of Medicinal Chemistry 28, 1874-1879 (1985), [26]) from the corresponding aminoaldehyde derived from the protected trans-4-hydroxy-L-proline and H-Lys(Z)-OtBu.HCl.

The purification of these compounds can be carried out by any technique known to those skilled in the art (W. Clark Still, Michael Kahn, and Abhijit Mitra J. Org. Chem. Rapid Chromatographic Technique for Preparative Separations with Moderate Resolution, J. Org. Chem., 1978, 43(14), 2923-2925 [14]).

The salts can be prepared according to techniques well known to those skilled in the art (Vogel's textbook of practical organic chemistry, Vth edition, 1989, Longman Scientific & Technical, publisher [34]).

Reversin 121 is a compound of formula (VII):

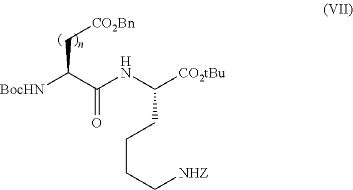

Reversin can be prepared by any process known to those skilled in the art, for example that described in document U.S. Pat. No. 6,297,216, or in the documents Sharom et al. ([23]) or Palyi et al. (Palyi, I. et al. Compounds for reversing drug resistance. Hungary patent (2001), [27]).

The compounds as defined above have a low IC₅₀ value. The IC₅₀ is defined as the concentration of compound which blocks or inhibits 50% of the chemotherapeutic agent efflux.

This value may be less than that of reversin 121. It is, for example, between 0.01±0.001 μM and 1.5±0.03 μM. For example, the compound as previously defined has an IC₅₀ of approximately 0.22±0.03 μM.

The tests for determining the activity of the compounds of the invention and that of reversin 121 are well known to those skilled in the art. It is possible to use, for example, the tests described in example 2 hereinafter.

The compounds of the invention exhibit non-significant inhibition of MRP1 and of BCRP. For example, the compounds of the invention can exhibit less than 15% inhibition of MRP1 or of BCRP, at 10 μM.

Advantageously, no sign of cytotoxicity is observed with the compounds of the invention at the pharmacologically active doses, which makes them compatible with use as medicaments.

For the purpose of the present invention, the term “medicament” is intended to mean any substance or composition that can be used in a mammal, i.e. humans or animals, which can be administered to them, for the purposes of establishing a medical diagnosis or of restoring, correcting or modifying their physiological functions by exerting a pharmacological, immunological or metabolic action. It may be a compound or composition of the invention administered as an adjuvant for a chemotherapy treatment of a patient, for which the treated patient's body develops a resistance. The medicament according to the invention can be intended for restoring or maintaining the activity of the chemotherapy treatment. The medicament according to the invention may make it possible to block, modulate or inhibit P-gp. The medicament according to the invention may make it possible to prevent or reduce chemotherapeutic agent efflux out of the target cells of the chemotherapy treatment. The compounds of the invention are therefore used as an adjuvant medicament for a chemotherapeutic agent.

For the purpose of the present invention, the term “animal” is intended to mean domestic animals, for example dogs, the Felidae such as cats and felines, rabbits, cattle, the ovine race, the Equidae, mammals, rodents, birds, reptiles, saurians, this list not being limiting.

For the purpose of the present invention, the term “mammal” is intended to mean any hot-blooded vertebrate animal, the heart of which has four cavities. This may for example involve humans, dogs, cats, rodents, cattle, the Equidae, the ovine race, this list not being limiting.

The compound of the invention can be administered to the mammal in an effective amount. For the purpose of the present invention, the term “effective amount” is intended to mean any amount of a compound of the invention which makes it possible to obtain the desired effect.

This amount can make it possible to improve one or more parameters characteristic of the pathological condition treated. This amount can also make it possible to obtain total or partial reduction of the efflux activity of P-gp. For example, an effective amount of the compound of the invention can make it possible to obtain a reduction of at least 50%, and advantageously at least 60% or 70% or 80% or 90% or 99% in the efflux activity of P-pg.

In other words, this amount can make it possible to reduce resistance to an active substance, in particular to a chemotherapeutic agent.

Advantageously, the effective amount of compound of structure (I) can be between 0.01 and 100 μM. Alternatively, the effective amount can be between 1 and 100 mg per kg of body weight per day.

The medicament can therefore be intended for reducing the activity of the ABCB1 efflux protein in a mammal.

The medicament can be intended for reducing the resistance to a pharmaceutically active substance.

The compound of the invention can be administered alone, or with a pharmaceutically active substance.

When it is administered with a pharmaceutically active substance, it can be described as an adjuvant to a treatment with this substance.

For example, the medicament can be an adjuvant to the chemotherapy treatment of a cancer or of an infection.

For the purpose of the present invention, the term “cancer” is intended to mean any pathological condition involving an abnormal/dysregulated growth of cells within a tissue of a living organism. By way of example, mention may be made of carcinomas, lymphomas, such as Hodgkin's disease and non-Hodgkin's lymphoma, blastomas, sarcomas, leukemias, and more particularly lung cancer, breast cancer, colon cancer: colon cancer, rectal cancer, pancreatic cancer, multiple myeloma, in particular bone marrow cancer, Kaposi's sarcoma, testicular cancer, adenocarcinoma, mesothelioma, glioma, melanoma, kidney cancer, prostate cancer, hepatocarcinoma, this list not being limiting.

For the purpose of the present invention, the term “infection” or “infectious disease” is intended to mean any pathological condition involving the invasion of cells within a tissue by a living organism. This living organism may be a virus, a bacterium, a parasite or a fungus. By way of example, mention may be made of tetanus, malaria, pneumonia, flu, aids, septicemia, rheumatic heart disease, appendicitis, peritonitis, tuberculosis, intestinal infections, viral hepatitis, this list not being limiting.

For the purpose of the present invention, the term “pharmaceutically active substance” is intended to mean any substance which has a pharmacological effect when it is administered to a human or to an animal.

For example, a pharmaceutically active substance may be a chemotherapeutic agent, for example antibiotic, antifungal, anticancer agent, anti-infective agent, but also any molecule of chemical origin.

For the purpose of the present invention, the term “chemotherapeutic agent” is intended to mean any chemical or synthetic substance used in the context of a therapy. In particular, it may be in an active substance intended for destroying cancer cells or cells infected with a pathogenic agent. It may then be a question of anticancer agents or anti-infective agents. By way of example, mention may be made of anthracyclines, topoisomerase inhibitors, antimetabolic agents such as antifolates, tyrosine kinase inhibitors, antivirals, for instance reverse transcriptase inhibitors, antiparasitics, antifungals, and in particular daunorubicin, doxorubicin, mitoxantrone, camptothecin and its derivatives, irinotecan, topotecan, indolocarbazoles, methotrexate, imatinib, gefitinib, zidovudine, lamivudine, abacavir, ivermectin, albendazole, oxfendazole, ketoconazole, this list not being limiting.

When the compound is administered alone, no chemotherapeutic agent is administered.

When the compound is administered with a chemotherapeutic agent, the compound of the invention and the chemotherapeutic agent can be administered simultaneously, in a sequenced manner, successively or spread out over time.

The compounds of the invention can be used for potentiating the effect, i.e. increasing the effect, of chemotherapeutic agents, in particular of anti-infective or anticancer agents. For example, these chemotherapeutic agents may become barely active or inactive with respect to resistant strains via an efflux mechanism. For the purpose of the present invention, the term “potentiation” is intended to mean that, when a compound of formula (I) and a chemotherapeutic agent, for example an anti-infective or anticancer agent, are combined, a therapeutic effect which is greater than that obtained with one or other only of the compounds is obtained. Advantageously, the therapeutic effect may be greater than the sum of the effects obtained separately.

An effective amount of the chemotherapeutic agent is preferably administered. Advantageously, the effective amount is an amount which does not make it possible to obtain destruction of the target cells when the compound of the invention is not administered with the chemotherapeutic agent. The effective amount of the therapeutic agent may be less than the effective amount of the chemotherapeutic agent when the agent is administered without the compound of the invention. Advantageously, the dose of chemotherapeutic agent administered can make it possible to obtain total or partial destruction of the target cells. Advantageously, an effective amount of the chemotherapeutic agent can make it possible to obtain destruction of at least 50%, and advantageously at least 60% or 70% or 80% or 90% or 99% of the target cells. Advantageously, the effective amount of compound of the chemotherapeutic agent may be between 0.1 and 100 μM relative to the total composition. Alternatively, the effective amount may be between 0.01 and 100 mg per kg of body weight per day.

The compound of the invention and the chemotherapeutic agent can be administered in a ratio which makes it possible to obtain total or partial inhibition of the efflux activity of P-gp and total or partial destruction of the target cells.

Various orders of administration or of treatment can be envisioned. The compound of the invention can be administered before, for example 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 8 weeks or 12 weeks before the administration of the chemotherapeutic agent.

Alternatively, the compound of formula as previously defined can be administered concomitantly, or after the administration of the chemotherapy agent, for example 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 8 weeks or 12 weeks after the administration of the chemotherapeutic agent.

Another subject of the invention relates to the compound of formula as previously defined, mixtures thereof, salts thereof and isomers thereof, as a medicament. This medicament can be used in humans or in animals.

The invention also relates to the compound of formula as previously defined, for treating a cancer or an infection. This compound can be used in humans or in animals.

The invention is also understood to mean a compound of formula as previously defined, for reducing the activity of the ABCB1 efflux protein in a human or an animal.

Another subject of the invention relates to a pharmaceutical composition comprising the compound of the invention.

The concentration of the compound of the invention may be, in the pharmaceutical composition, between 0.001 and 500 μM. For example, the concentration is between 0.001 and 50 μM, or between 0.001 and 1 μM.

The compound of the invention and the chemotherapeutic agent can be administered separately, each in a distinct pharmaceutical composition.

Alternatively, the compound of the invention and the chemotherapeutic agent can be administered in a single pharmaceutical composition. In other words, the medicament of the invention may be in the form of a single pharmaceutical composition combining, in the same formulation, (i) the compound of the invention and (ii) a chemotherapeutic agent as previously defined. Such a pharmaceutical composition can be used in humans or in animals.

The pharmaceutical composition also comprises a pharmaceutically acceptable carrier, advantageously chosen according to the pharmaceutical form and the mode of administration desired. The pharmaceutically acceptable carriers that can be used are the carriers that are known to those skilled in the art. By way of example, mention may be made of gelatin, starch, lactose, magnesium stearate, talc, gum arabic or any analog.

The pharmaceutical composition has the advantage of not inducing significant inhibition of MRP1 and of BCRP.

The pharmaceutical composition has the following advantages: a specificity for P-gp, an affinity for P-gp which is better than micromolar (i.e. less than or equal to 1 micromolar), for example up to 7 times better than that of reversin 121 for P-gp, a better level of inhibition than that of reversin 121, few or no adverse effects and no competition.

The mode of administration of the pharmaceutical composition or of the medicament may be oral, sublingual, subcutaneous, intramuscular, intravenous, topical, intratracheal, intranasal, transdermal, rectal or intraocular.

The administration form for the composition or for the medicament comprises all forms that are compatible with the desired objective. These administration forms are known to those skilled in the art, and include tablets, for example film-coated, coated or scored tablets, gel capsules, powders, granules, oral suspensions or solutions, creams, ointments, lotions or eye lotions, syrups, emulsions, this list not being limiting.

The form of the medicament or of the pharmaceutical composition may be a form suitable for prolonged or delayed activity. The form may be suitable for continuous release of a predetermined amount of compound of the invention and, optionally, of chemotherapeutic agent.

The formulations, in particular the tablets, gel capsules or granules, may be coated with sucrose, or with a cellulose derivative.

For example, the gel capsules may be prepared by mixing the active ingredient with a diluent and pouring the mixture obtained into gel capsules, for example soft or hard gel capsules.

The liquid forms containing the medicament or the pharmaceutical composition of the invention may have any suitable liquid as carrier. Such carriers are known to those skilled in the art, and mention may, for example, be made of water, solvents, in particular organic solvents such as glycerol or glycols, and also mixtures thereof, in varied proportions.

A preparation in the form of a syrup, of an elixir or of drops may contain a sweetener, for example a calorie-free sweetener, an antiseptic such as methylparaben or propylparaben, a flavoring and a suitable colorant.

The water-dispersible powders or granules may contain the active ingredient as a mixture with dispersants or wetting agents, or suspension agents, for instance polyvinylpyrrolidone, or with sweeteners or flavor enhancers.

Another subject of the invention is the use of the compound of the invention for treating pathological conditions. By way of example, mention may be made of cancers or infections, in humans or animals.

Another subject of the invention relates to the use of a compound of formula (I) for inhibiting the ABCB1 efflux pump in vitro. For example, a compound of formula (I) can be used to screen molecules, in particular chemotherapeutic agents.

Another subject of the invention relates to a method of therapeutic treatment, in particular for a patient requiring same, comprising the administration of a compound of structure (I) or a pharmaceutically acceptable salt thereof.

Other advantages may become further apparent to those skilled in the art on reading the examples below, illustrated by means of the appended figures, given by way of illustration.

### BRIEF DESCRIPTION OF THE FIGURES

FIG. 1 shows the impact of the expression of P-gp and of BCRP on the percentages of life expectancy and of remission for acute myeloid leukemias ([12]).

FIG. 2 shows compounds Nos 1, 2, 3, 4, 5 and 6 and their effect on the efflux function of P-gp. Reversin 121 is the reference compound ([23], [27]). The P-gp inhibition percentages are indicated, as is the concentration for 50% maximum effect, IC₅₀.

FIGS. 3A, B, C, D and E show the (percentage) inhibition of the efflux of mitoxantrone (MTX), which is a P-gp-transported anticancer agent, respectively by reversin 121 ([27]), compound No. 3, compound No. 5, compound No. 4, and compound No. 6. Each IC₅₀ value is obtained by mathematical adjustment of the experimental points and recorded in table 2.

FIG. 4 shows the chemosensitization to mitoxantrone of control NIH3T3 cells (circles, squares) or NIH3T3 cells expressing P-gp (triangles, diamonds) with compound No. 6. Increasing concentrations (μM) of mitoxantrone (MTX) are applied to NIH3T3 cells expressing P-gp or not expressing P-gp, in the presence or absence of compound No. 6. The cell survival rate is expressed as a percentage.

FIGS. 5A and 5B show, respectively, the cytotoxicity of compounds No. 4 and No. 6, applied at increasing concentrations (μM) to NIH3T3 cells expressing (squares) or not expressing (circles) P-gp.

FIGS. 6A and 6B show the kinetics of inhibition of daunorubicin efflux produced by P-gp, with compound No. 6. The intracellular daunorubicin fluorescence is measured in the presence of increasing concentrations of No. 6, respectively 0 (circles), 0.12 μM (triangles), 0.22 μM (squares), 0.44 μM (diamonds) and 10 μM (upward-pointing triangles), corresponding to 0, 25, 50, 75 and 100% efflux inhibition effectiveness. FIG. 6A: direct representation, FIG. 6B, Lineweaver & Burke double reciprocal representation.

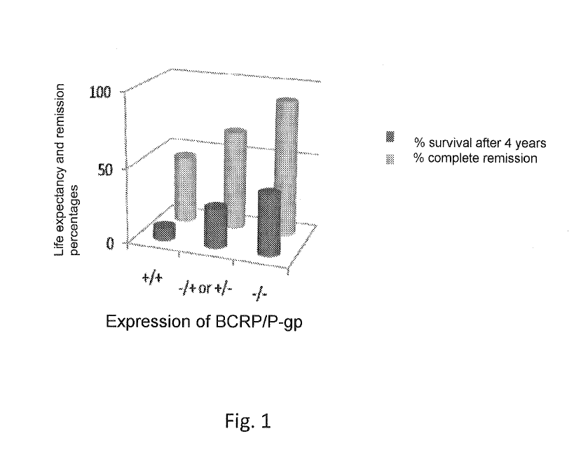

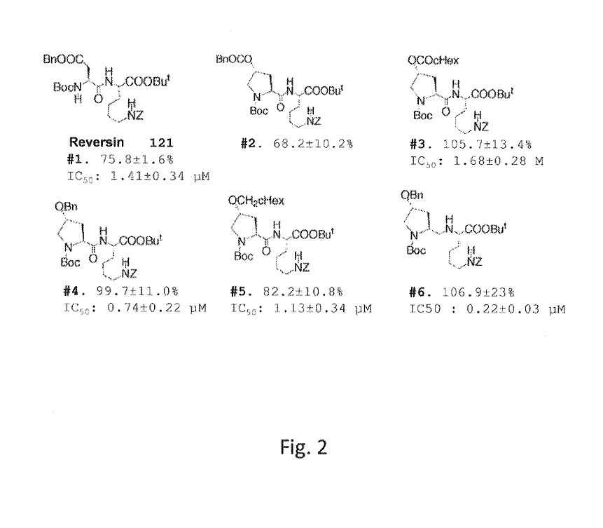

### EXAMPLES

### Example 1

### Preparation of Compounds Nos 2, 3, 4, 5 and 6

In the examples which follow, the following abbreviations are used: h means hour, a.t. means ambient temperature, asym means asymmetric, EtOAc means ethyl acetate, Boc means Cert-butyloxycarbonyl, (Boc)₂O means di-tert-butyl dicarbonate, Bu means butyl, DMF means N,N-dimethylformamide, PE means petroleum ether, ESI means electrospray ionization, Et means ethyl, Et₂O means diethyl ether, HR means high resolution, Me means methyl, Ph means phenyl, eq means equivalent, Hyp means 4-hydroxyproline, Lys means lysine, IR means infrared, NMR means nuclear magnetic resonance, MS means mass spectrometry, TBTU means 2-(1-hydroxybenzotriazol-1-yl)-1,1,3,3-tetrahydrouronium tetrafluoroborate.

### 1) Synthesis of N^{α}-Boc-trans-4-Hyp(COR)-Lys(Z)-OtBu

The synthesis is carried out using commercial trans-4-hydroxy-L-proline (Novabiochem ref. 04-10-0020), the acid and amine functions of which are pre-protected. Next, esterification of the alcohol, followed by deprotection of the acid and coupling with the commercial diprotected lysine derivative (Novabiochem ref. 04-12-5122), results in the target restricted analog.

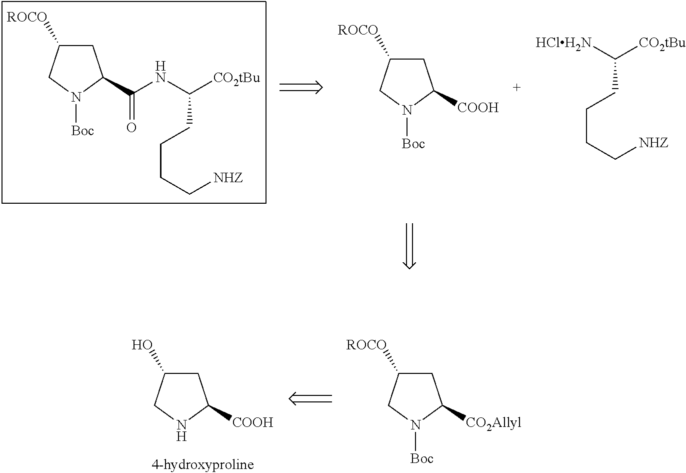

### 1.1) Synthesis of N^{α}-Boc-trans-4-Hyp-OAllyl

The derivative 74 is prepared with a yield of 97% according to the procedure described by Carrasco and Brown (Carrasco M. R., Brown R. T. J. Org. Chem., 2003, 68(23), 8853-8858, [31]) on L-homoserine.

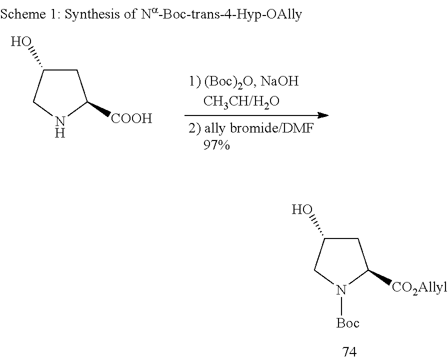

Allyl ester of (4R)-hydroxy-N-(tert-butyloxycarbonyl)pyrrolidin-(2S)-oic acid 74

500 mg of commercial trans-4-hydroxy-L-proline (3.816 mmol; 500 mg) and 170 mg of NaOH (4.2 mmol; 1.1 eq) are dissolved in 5 ml of water and 5 ml of CH₃CN. 1.24 g of (Boc)₂O (5.7 mmol; 1.5 eq) are then added and the solution is stirred over night at ambient temperature. The solvents are evaporated off and the oily residue obtained is triturated with Et₂O, dried under vacuum, and then dissolved in 12 ml of DMF. The resulting solution is treated with 0.364 ml of allyl bromide (4.2 mmol; 1.1 eq) and the reaction is stirred over night at ambient temperature. The DMF is evaporated off and the residue obtained is dissolved in EtOAc. This organic phase is washed three times with 25 ml of a saturated solution of NaHCO₃, once with 25 ml of water, twice with 25 ml of a 0.1 M solution of KHSO₄ and once with 25 ml of a saturated solution of NaCl. The organic phase is dried over anhydrous Na₂SO₄, filtered and concentrated. The product (3.69 mmol; 1 g) is obtained in the form of a colorless oil which does not require purification.

IR (NaCl; film): 3436 (v_{OH}); 2978 and 2936 (v_{CH}); 1748 and 1682 (v_{C=O}); 1415 (v_{C-C-O asym}); 1368 (v_{CH3}t-Bu); 1159 (v_{O-C-C asym}).

¹H NMR (CDCl₃; 300 MHz): 1.42 and 1.46 (9H, 2s, C(CH₃)₃ Boc 1st and 2nd conf.); 2.09 (1H, m, CH_{β}); 2.31 (1H, m, CH_{β}); 3.55 (2H, m, CH₂N); 4.40-4.51 (2H, m, CH_{α} and CHOH); 4.65 (2H, d, J=5.6 Hz, CH₂—CH═CH₂); 4.26 (1H, dd, J=0.7 Hz and J=10.1 Hz, CH₂—CH═CH₂); 5.84 (1H, dd, J=0.9 Hz and J=18.2 Hz, CH₂—CH═CH₂); 5.91 (1H, m, CH₂—CH═CH₂).

### 1.2) Synthesis of N^{α}-Boc-trans-4-Hyp(COR)—O Allyl

The alcohol 74 is esterified with phenylacetyl chloride or cyclohexanoyl chloride. There is total reaction in the case of the compound 75a using an excess of cyclohexanoyl chloride and two equivalents of triethylamine. The two esters 75a and 75b are obtained with yields of 43 and 84%, respectively.

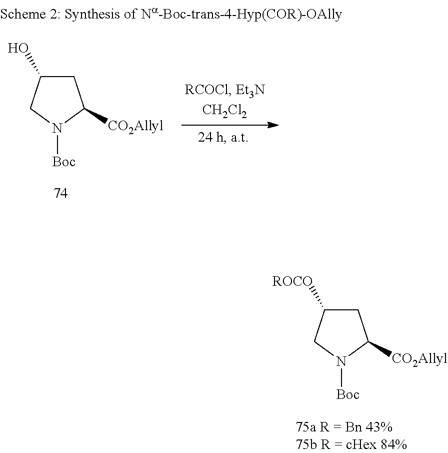

### Allyl ester of (4R)-(benzylcarbonyloxy)-N-(tert-butyl-oxycarbonyl)pyrrolidin-(2S)-oic acid 75a

156 μl of triethylamine (1.107 mmol; 1.5 eq) and 120 μl of phenylacetyl chloride (0.885 mmol; 1.2 eq) at 0° C. are added to a solution of N^{α}-Boc-trans-4-Hyp-OAllyl 74 (0.738 mmol; 200 mg) in 7 ml of CH₂Cl₂. After stirring over night at ambient temperature, 15 ml of water are added and the reaction is extracted with 20 ml of CH₂Cl₂. The organic phase is washed with water, dried over anhydrous Na₂SO₄, filtered and concentrated. The oily yellowish residue obtained is purified by silica gel column chromatography (EtOAc/PE 20:80). The product (0.320 mmol; 123 mg) is obtained in the form of a yellowish oil.

IR (NaCl; film): 3065 and 3031 (v_{φCH}); 2978 and 2933 (v_{CH}); 1744 and 1704 (v_{C=O}); 1455 (v_{Φc=c}); 1403 and 1367 (δ_{CH3}t-Bu); 1258 (v_{C-C-O asym}); 1157 (v_{O-C-C asym}); 931 (δ_{CH} vinyl).

¹H NMR (CDCl₃; 300 MHz): 1.44 and 1.46 (9H, 2s, C(CH₃)₃ Boc 1st and 2nd conf.); 2.20 (1H, m, CH_{β}); 2.38 (1H, m, CH_{β}); 3.62-3.76 (4H, s+m, CH₂Ph and CH₂N); 4.32 and 4.43 (1H, 2t, J=8.0 Hz and J=7.7 Hz, CH_{α} 1st and 2nd conf.); 4.65 (2H, m, CH₂—CH═CH₂); 5.22-5.38 (3H, m, CH₂—CH═CH₂) and CHOCOBn); 5.92 (1H, m, CH₂—CH═CH₂), 7.33 (5H, m, Ph).

### Allyl ester of (4R)-(cyclohexylcarbonyloxy)-N-(tert-butyloxycarbonyl)pyrrolidin-(2S)oic acid 75b

270 μl of triethylamine (1.92 mmol; 2 eq) and 224 μl of cyclohexanoyl chloride (1.632 mmol; 1.7 eq) at 0° C. are added to a solution of N^{α}-Boc-trans-4-Hyp-OAllyl 74 (0.960 mmol; 260 mg) in 8 ml of CH₂Cl₂. After stirring over night at ambient temperature, 15 ml of water are added and the reaction is extracted with 20 ml of CH₂Cl₂. The organic phase is washed with water, dried over anhydrous Na₂SO₄, filtered and concentrated. The oily yellowish residue obtained is purified by silica gel column chromatography (EtOAc/PE 20:80). The product (0.808 mmol; 308 mg) is obtained in the form of a colorless oil.

IR (NaCl; film): 2977 and 2934 (v_{CH}); 1733, 1705 and 1699 (v_{C=O}); 1403 and 1368 (v_{CH3}t-Bu); 1248 (v_{C-C-O asym}); 1163 (v_{O-C-C asym}); 929 (v_{CH} vinyl).

¹H NMR (CDCl₃; 300 MHz): 1.21-1.38 (4H, m, 2CH₂ cHex); 1.44 and 1.47 (9H, s, C(CH₃)₃ Boc 1st and 2nd conf.); 1.61-1.91 (6H, m, 3CH₂ cHex); 2.14-2.43 (3H, m, CH_{2α} and CHCO cHex); 3.63 (2H, m, CH₂N); 4.35 and 4.45 (1H, 2t, J=8.0 Hz and J=7.7 Hz, CH_{α} 1st and 2nd conf.); 4.66 (2H, m, CH₂—CH═CH₂); 5.23-5.38 (3H, m, CH₂—CH═CH₂ and CH—O Hyp); 5.92 (1H, m, CH₂—CH═CH₂).

### 1.3) Synthesis of N^{α}-Boc-trans-4-Hyp(COR)—OH

Cleavage of each of the two allyl esters 75a and 75b according to the procedure of Carrasco and Brown ([31]) results, respectively, in the acid 76a with a quantitative yield and in the acid 76b with a yield of 95%.

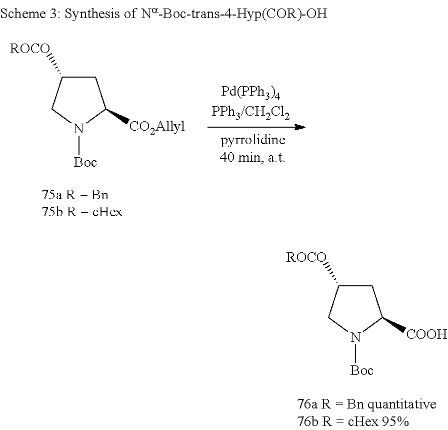

### (4R)-(benzylcarbonyloxy)-N-(tert-butyloxycarbonyl)pyrrolidin-(2S)-oic acid 76a

4 mg of PPh₃ (0.01555 mmol; 0.05 eq) and 27 μl of pyrrolidine (0.3265 mmol; 1.05 eq) are added to a solution of N^{α}-Boc-trans-4-Hyp(COBn)-OAllyl 75a (0.311 mmol; 121 mg) in 3 ml of CH₂Cl₂, and then the mixture is stirred at ambient temperature under an argon atmosphere. 9 mg of Pd(0) (0.007775 mmol; 0.025 eq) are then added and the mixture is stirred for 40 minutes. The solvent is evaporated off and the residue obtained is dissolved in EtOAc. This organic phase is washed three times with a 0.1 M solution of KHSO₄ and once with a saturated solution of NaCl, and then dried over anhydrous Na₂SO₄, filtered and concentrated. The oily yellow residue obtained is purified by silica gel column chromatography (EtOAc/PE 50:50 then with the same eluent +0.1% of formic acid). The product (0.309 mmol; 108 mg) is obtained in the form of a yellowish oil.

IR (NaCl; film): 2980 (v_{COOH}), 1732 (v_{C=O}); 1455 (v_{φC=C}), 1369 (δ_{CH3} t-Bu); 1257 (v_{C-C-O asym}), 1160 (v_{O-C-C asym}).

¹H NMR (CDCl₃; 300 MHz): 1.46 and 1.48 (9H, 2s, C(CH₃)₃ Boc 1st and 2nd conf.); 2.40 (2H, m, CH_{2β}); 3.60-3.72 (4H, m+s, CH₂N and CH₂Ph); 4.32 and 4.44 (1H, 2t, J=7.9 Hz and J=7.9 Hz, CH_{α} 1st and 2nd conf.); 5.29 (1H, m, CHOCOBn); 7.32 (5H, m, Ph).

### (4R)-(cyclohexylcarbonyloxy)-N-(tert-butyloxycarbonyl)pyrrolidin-(2S)-oic acid 76b

This product is prepared according to the same procedure as for the product 76a using N^{α}-Boc-trans-4-Hyp(COcHex)-OAllyl 75b (0.753 mmol; 287 mg). The oily yellow residue obtained is purified by silica gel column chromatography (EtOAc/PE 50:50 then with the same eluent +0.1% of formic acid). The product (0.718 mmol; 245 mg) is obtained in the form of a yellowish oil.

IR (NaCl; film): 3446 (v_{COOH}), 2978 and 2933 (v_{CH}), 1732 (v_{C=O}); 1368 (δ_{CH3} t-Bu); 1249 (v_{C-C-O asym}).

¹H NMR (CDCl₃; 300 MHz): 1.25-1.41 (4H, m, 2CH₂1 cHex); 1.45 and 1.49 (9H, s, C(CH₃)₃ Boc 1st and 2nd conf.); 1.63-1.90 (6H, m, 3CH₂ cHex); 2.23-2.52 (3H, m, CH_{2β} and CHCO cHex); 3.65 (2H, m, CH₂N); 4.36 and 4.50 (1H, 2t, J=8.0 Hz and J=7.9 Hz, CH_{α} 1st and 2nd conf.); 5.29 (1H, d, J=15.4 Hz, CH—O Hyp); 6.30-7.80 (1H, M, COOH).

### 1.4) Synthesis of N^{α}-Boc-trans-4-Hyp(COR)-Lys(Z)-OtBu

The two acids 76a and 76b are coupled in two one-pot steps with the commercial H-Lys(Z)-OtBu.HCl (Novabiochem ref. 04-12-5122) according to the method of Lai et al. ([25]).

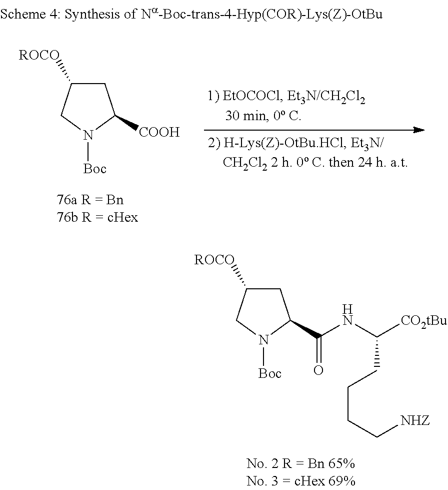

The overall yield of each of the two restricted derivatives, compounds No. 2 and No. 3, is 27% and 53%, respectively, in four steps starting from the commercial trans-4-hydroxy-L-proline (Novabiochem ref. 04-10-0020).

### N^{α}-Boc-trans-L-4-Hyp(COBn)-L-Lys(Z)-OtBu 77a

The N^{α}-Boc-trans-4-Hyp(COBn)-OH 76a (0.30 mmol; 102 mg) is dissolved in 4 ml of CH₂Cl₂ under argon. The solution is cooled to 0° C. and then 50 μl of triethylamine (0.33 mmol; 1.1 eq) are added dropwise for 5 minutes. 40 μl of ethyl chloroformate (0.33 mmol; 1.1 eq) are added dropwise for 5 minutes. The mixture is stirred at 0° C. for 30 minutes, and then a solution of commercial H-Lys(Z)-OtBu.HCl (0.30 mmol; 112 mg) and of 100 μl of triethylamine (0.66 mmol; 2.2 eq) in 4 ml of CH₂Cl₂ at 0° C. is added over the course of 5 minutes. The solution is stirred at 0° C. for two hours and then at ambient temperature over night. 5 ml of CH₂Cl₂ are added and the organic phase is washed twice with 10 ml of a saturated solution of NaHCO₃ and twice with a 1M solution of HCl. The organic phase is dried over anhydrous Na₂SO₄, filtered and concentrated. The oily yellow residue obtained is purified by silica gel column chromatography (EtOAc/PE 50:50). The product (0.195 mmol; 130 mg) is obtained in the form of a colorless oil. Elemental analysis: Found C, 64.93%; H, 7.37%; N, 6.06%. C₃₆H₄₉N₃O₉ required C, 64.75%; H, 7.40%; N, 6.29%.

IR (NaCl; film): 3329 (v_{NH}); 3065 and 3033 (v_{φCH}); 2978 and 2933 (v_{CH}); 1695 (v_{C=O} amide); 1455 (v_{φC=C}); 1394 and 1368 (δ_{CH3} t-Bu); 1251 (v_{C-C-O asym}), 1158 (v_{O-C-C asym}).

¹H NMR (CDCl₃; 300 MHz): 1.44 and 1.49 (9H, 2s, C(CH₃)₃ Boc 1st and 2nd conf.); 1.34-1.69 (6H, m, 3CH₂ Lys); 2.20 (1H, m, CH_{β}); 2.45 (1H, m, CH_{β}); 3.17 (2H, m, CH₂NH); 3.55-3.63 (4H, m+s, CH₂N and CH₂Ph); 4.28 (1H, m, CH_{α}); 4.44 (1H, m, CH_{α}); 5.10 (2H, s, CH₂Ph Z); 5.25 (1H, m, CH—O Hyp); 7.25-7.37 (10H, m, 2Ph).

¹³C NMR (CDCl₃; 75 MHz): 22.62 (CH₂ Lys); 28.38 and 28.64 (6CH₃ Boc and t-Bu); 29.44 and 30.11 (2CH₂ Lys); 32.38 (CH_{2β}); 40.96 and 41.72 (CH₂NH and CH₂Ph Hyp); 52.91 (CH₂N Hyp); 58.97 (CH_{α}); 60.82 (CH_{α}); 66.94 (CH₂Ph Z); 73.68 (CHO Hyp); 81.16 (C—O t-Bu); 82.47 (C—O t-Bu); 127.66, 128.89, 129.07 and 130.0 (10CH 2Ph); 133.94 (Cq Ph); 137.07 (Cq Ph); 155.50 and 156.89 (2CO Boc and Z); 171.23, 171.37 and 171.59 (CO amide and 2CO ester).

MS (ESI; positive mode): 1357.1 [2M+Na]⁺; 690.2 [M+Na]⁺.

HR MS (ESI; positive mode): 690.33599 [M+Na]⁺ (calc. 690.3367).

### N^{α}-Boc-trans-L-4-Hyp(COcHex)-L-Lys(Z)-OtBu 77b

This dipeptide is prepared according to the same procedure as for the product 77a using the N^{α}-Boc-trans-4-Hyp(COcHex)-OH 76b (0.642 mmol; 219 mg) and the commercial H-Lys(Z)-OtBu.HCl (0.642 mmol; 240 mg). The pasty yellowish residue obtained is purified by silica gel column chromatography (EtOAc/PE 40:60). The product (0.443 mmol; 292 mg) is obtained in the form of a viscous yellow oil.

Elemental analysis: Found C, 63.95%; H, 8.20%; N, 6.30%. C₃₅H₅₃N₃O₉ required C, 63.71%; H, 8.10%; N, 6.37%.

IR (NaCl; film): 3350 (v_{NH}); 2933 (v_{CH}); 1668 (v_{C=O} amide); 1454 (v_{φC=C}); 1394 (δ_{CH3} t-Bu); 1248 (v_{C-C-O asym}); 1160 (v_{O-C-C asym}).

¹H NMR (CDCl₃; 300 MHz): 1.44 and 1.47 (9H, 2s, C(CH₃)₃ Boc 1st and 2nd conf.); 1.23-1.88 (16H, m, 2CH₂ Lys and 5CH₂ cHex); 2.18-2.54 (3H, m, CH_{2β} and CHCO cHex); 3.19 (2H, m, CH₂NH); 3.56 (2H, M, CH₂N); 4.36 (1H, m, CH_{α}); 4.45 (1H, m, CH_{α}); 5.10 (2H, system AB, J=12.4 Hz, CH₂Ph); 5.22 (1H, M, CH—O Hyp); 7.05 (1H, M, NH); 7.34 (5H, m, Ph).

¹³C NMR (CDCl₃; 75 MHz): 21.46, 22.19, 22.64, 29.43, 30.08, 32.35, 32.94 and 34.68 (5CH₂ cHex and 3CH₂ Lys); 28.37 and 28.64 (6CH₃ Boc and t-Bu); 37.32 (CH_{2β}); 43.35 (CHCO cHex); 53.11 (CH₂NH); 59.04 (CH_{α}); 60.41 (CH_{α}); 60.80 (CH₂N); 72.81 (CH—O Hyp); 73.67 (CH₂Ph); 81.11 (C—O t-Bu); 82.43 (C—O t-Bu); 128.16, 128.43, 128.51 and 128.87 (5CH Ph); 137.06 (Cq Ph); 155.67 and 156.89 (2CO Boc and Z); 171.30, 171.95 and 175.87 (CO amide and 2CO ester).

MS (ESI; positive mode): 1341.2 [2M+Na]⁺; 682.2 [M+Na]⁺; 660.0 [MH]⁺.

HR MS (ESI; positive mode): 682.36780 [M+N]⁺ (calc. 682.3680).

### 2) Synthesis of N^{α}-Boc-trans-4-Hyp(Bn)-Lys(Z)-OtBu

This synthesis is carried out by peptide coupling of the two commercial derivatives, N^{α}-BOC-trans-4-Hyp(Bn)-OH (Bachem ref. 54631-81-1) and H-Lys(Z)-OtBu.HCl (Novabiochem ref. 04-12-5122) in two one-pot steps and under the conditions previously used ([25]) for this type of coupling. The yield of the dipeptide No. 4 is 70%.

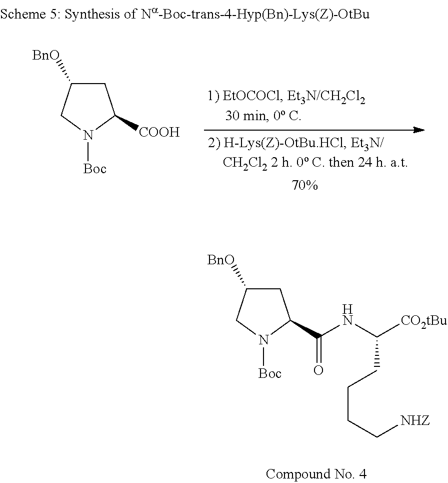

### N^{α}-Boc-trans-L-4-Hyp(Bn)-L-Lys(Z)-OtBu 78

This dipeptide is prepared according to the same procedure as for the product 77a using the commercial N^{α}-Boc-trans-4-Hyp(Bn)-OH (0.623 mmol; 200 mg) and the commercial H-Lys(Z)-OtBu.HCl (0.623 mmol; 232 mg). The oily yellow residue obtained is purified by silica gel column chromatography (EtOAc/PE 40:60). The product (0.438 mmol; 280 mg) is obtained in the form of a more or less colorless oil.

Elemental analysis: Found C, 65.77%; H, 7.66%; N, 6.50%. C₃₅H₄₉N₃O₈ required C, 65.71%; H, 7.72%; N, 6.57%.

IR (NaCl; film): 3325 (v_{NH}); 3065 and 3033 (v_{φCH}); 2977 and 2934 (v_{CH}); 1679 (v_{C=O} amide); 1455 (v_{φC=C}); 1394 and 1367 (v_{CH3} t-Bu); 1251 (v_{C-C-O asym}); 1160 (v_{O-C-C asym}).

¹H NMR (CDCl₃; 300 MHz): 1.35 and 1.38 (9H, 2s, C(CH₃)₃ Boc 1st and 2nd conf.); 1.23-1.78 (6H, m, 3CH₂ Lys); 2.07 (1H, m, CH_{β}); 2.32 (1H, m, CH_{β}); 3.09 (2H, m, CH₂NH); 3.44 (2H, M, CH₂N); 4.24-4.37 (2H, m, 2CH_{α}); 4.41 (2H, d, J=3.7 Hz, CH₂Ph Hyp); 4.96-5.05 (3H, m, CH—O Hyp and CH₂Ph Z); 6.52 (1H, M, NH); 6.97 (1H, M, NH); 7.20-7.27 (10H, m, 2Ph).

¹³C NMR (CDCl₃; 75 MHz): 22.67 (CH₂ Lys); 28.39 and 28.70 (6CH₃ Boc and t-Bu); 29.45 and 32.37 (2CH₂ Lys); 34.72 (CH_{2β}); 40.97 (CH₂NH); 52.39 (CH₂N); 59.24 (CH_{α}); 60.42 (CH_{α}); 66.89 (CH₂Ph Hyp); 71.64 (CH₂Ph Z); 77.33 (CH—O Hyp); 80.84 (C—O t-Bu); 82.34 (C—O t-Bu); 128.03, 128.18, 128.50 and 128.87 (10CH 2Ph); 137.09 (Cq Ph); 138.28 (Cq Ph); 155.76 and 156.89 (2CO Boc and Z); 171.62 and 172.72 (CO amide and CO ester).

MS (ESI; positive mode): 662.3 [M+Na]⁺; 540.2 [MH]-isobutene—CO₂.

HR MS (ESI; positive mode): 662.34155 [M+Na]⁺ (calc. 662.3417).

### 3) Synthesis of N^{α}-Boc-trans-4-Hyp(CH₂cHex)-Lys(Z)-OtBu

The synthesis strategy involves a peptide coupling reaction between N^{α}-Boc-trans-4-Hyp(CH₂cHex)-OH and the commercial H-Lys(Z)-OtBu.HCl (Novabiochem ref. 04-12-5122). The cyclohexyl methyl ether of N^{α}-Boc-trans-4-Hyp-OH can be produced by catalytic hydrogenation of commercial N^{α}-Boc-trans-4-Hyp(Bn)-OH benzyl ether (Bachem ref. 54631-81-1).

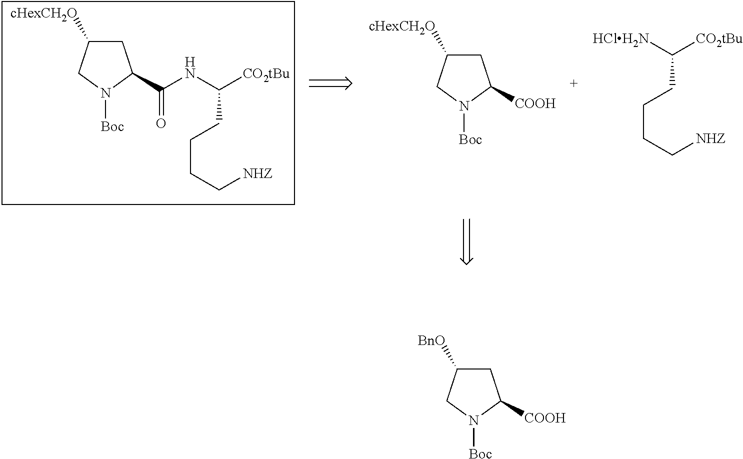

### 3.1) Synthesis of N^{α}-Boc-trans-4-Hyp(CH₂cHex)-OH

Seko et al. (Seko T., Kato M., Kohno H., Ono S., Hashimura K., Takimizu H., Nakai K., Maegawa H., Katsube N., Toda M. Bioorg. Med. Chem., 2003, 11(8), 1901-1913, [28]) describe the synthesis of N^{α}-Boc-L-Ser(CH₂cHex)-OH by catalytic hydrogenation of N^{α}-Boc-L-Ser(Bn)-OH in the presence of rhodium on alumina (Rh—Al₂O₃) in isopropanol. The cyclohexyl methyl ether of the N^{α}-protected serine is obtained with a yield of 86% after recrystallization from hexane.

The reaction is carried out under the same conditions on the commercial N^{α}-Boc-trans-4-Hyp(Bn)-OH (Bachem ref. 54631-81-1), and the cyclohexyl methyl ether 79 is obtained with a quantitative yield.

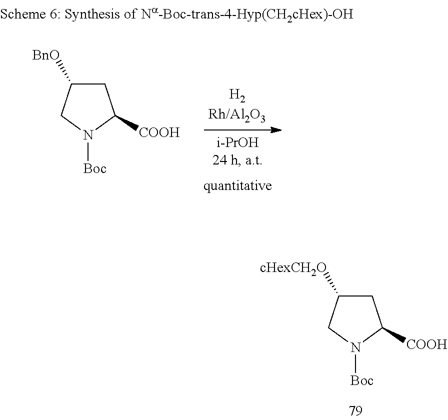

### (4R)-(cyclohexylmethyloxy)-N-(tert-butyloxycarbonyl)pyrrolidin-(2S)-oic acid 79

A mixture of commercial N^{α}-Boc-trans-4-Hyp(Bn)-OH (0.623 mmol; 200 mg) and of Rh/Al₂O₃ (5%) (0.018 mmol; 37 mg; 0.028 eq) in 4 ml of isopropanol is stirred at ambient temperature and under a hydrogen atmosphere over night. The mixture is filtered in order to remove the catalyst and the filtrate is concentrated. The product (0.62 mmol; 203 mg) is obtained in the form of a more or less colorless oil which does not require purification.

IR (NaCl; film): 3418 (v_{COOH}); 2974 and 2924 (v_{CH}); 1705 and 1682 (v_{C=O}); 1368 (δ_{CH3} t-Bu); 1257 (v_{C-C-O asym}); 1163 (v_{O-C-C asym}).

¹H NMR (CDCl₃; 300 MHz): 1.17-1.27 (6H, m, 3CH₂ cHex); 1.43 and 1.50 (9H, s, C(CH₃)₃ Boc 1st and 2nd conf.); 1.65-1.76 (5H, m, 2CH₂ and CH cHex); 2.16 (1H, m, CH_{β}); 2.39 (1H, m, CH_{β}); 3.21 (2H, m, CH₂N); 3.54 (2H, m, CH₂O); 4.05 (1H, m, CH—O); 4.34 and 4.46 (1H, 2t, J=7.7 Hz and J=7.3 Hz, CH_{α}); 5-5.90 (1H, M, COOH).

### ii—Synthesis of N^{α}-Boc-trans-4-Hyp(CH₂cHex6-Lys(Z)-OtBu

The acid 79 is coupled with the H-Lys(Z)-OtBu.HCl in two one-pot steps under the conditions for synthesis of the compound 77a. This coupling resulted in the dipeptide 80 with a yield of 67%.

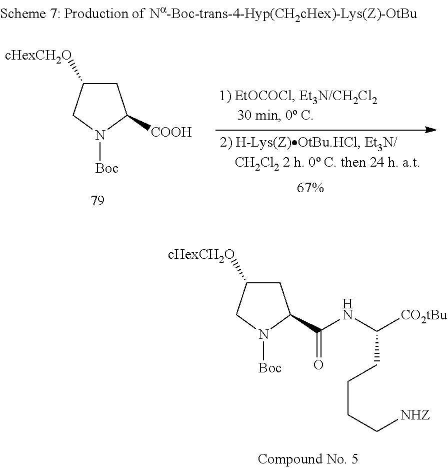

### N^{α}-Boc-trans-L-4-Hyp(CH2cHex)-L-Lys(Z)-OtBu 80

This dipeptide is prepared according to the same procedure as for the product 77a using the N^{α}-Boc-trans-4-Hyp(CH₂cHex)-OH 79 (0.62 mmol; 203 mg) and the commercial H-Lys(Z)-OtBu.HCl (0.62 mmol; 231 mg). The oily yellowish residue obtained is purified by silica gel column chromatography (EtOAc/PE 40:60). The product (0.414 mmol; 267 mg) is obtained in the form of a yellow oil.

Elemental analysis: Found C, 65.34%; H, 8.78%; N, 6.40%. C₃₅H₅₅N₃O₈ required C, 65.09%; H, 8.58%; N. 6.51%.

IR (NaCl; film): 3325 (v_{NH}); 3066 and 3034 (v_{φCH}); 2977 and 2925 (v_{CH}); 1681 (v_{C=O} amide); 1454 (v_{φC=C}); 1394 and 1368 (δ_{CH3} t-Bu); 1248 (v_{C-C-O asym}); 1159 (v_{O-C-C asym}).

¹H NMR (CDCl₃; 300 MHz): 1.44 and 1.47 (9H, 2s, C(CH₃)₃ Boc 1st and 2nd conf.); 1.12-1.89 (17H, m, 3CH₂ Lys, 5CH₂ and CH cHex); 2.06 (1H, m, CH_{β}); 2.34 (1H, m, CH_{β}); 3.13-3.21 (4H, m, 2CH₂N); 3.46 (2H, M, CH₂O Hyp); 3.95 and 4.04 (1H, 2M, CH_{α} 1st and 2nd conf.); 4.31 (1H, m, CH_{α}); 4.43 (1H, m, CH—O Hyp); 5.10 (3H, M+s, NH and CH₂Ph); 7.06 (1H, d, J=7.9 Hz, NH); 7.36 (5H, s, Ph).

^{—}C NMR (CDCl₃; 75 MHz): 21.43, 22.30, 22.68, 29.43, 30.08, 30.37, 30.43 and 32.39 (5CH₂ cHex and 3CH₂ Lys); 28.38 and 28.68 (6CH₃ Boc and t-Bu); 34.73 (CH_{2β}); 38.46 (CH cHex); 41.0 (CH₂NH); 52.33 (CH₂N); 52.92 (CH_{α}); 59.25 (CH_{α}); 66.90 (CH₂O Hyp); 75.48 (CH₂Ph); 77.59 (CH—O Hyp); 80.71 (C—O t-Bu); 82.29 (C—O t-Bu); 128.46 and 128.86 (5CH Ph); 137.10 (Cq Ph); 155.89 and 156.86 (2CO Boc and Z); 171.61 and 172.87 (CO amide and CO ester).

MS (ESI; positive mode): 1313.3 [2M+Na]⁺; 668.3 [M+Na]⁺; 646.1 [MH]⁺.

HR MS (ESI; positive mode): 668.38834 [M+Na]⁺ (calc. 668.3887).

### 4) Synthesis of N^{α}-Boc-trans-4-Hyp(Bn)-ψ(CH₂NH)-Lys(Z)-OtBu

The aminomethyl analog of compound No. 4 is synthesized by means of a reductive amination reaction between the aldehyde derived from doubly protected trans-4-hydroxy-L-proline and protected lysine according to the retrosynthetic scheme represented below.

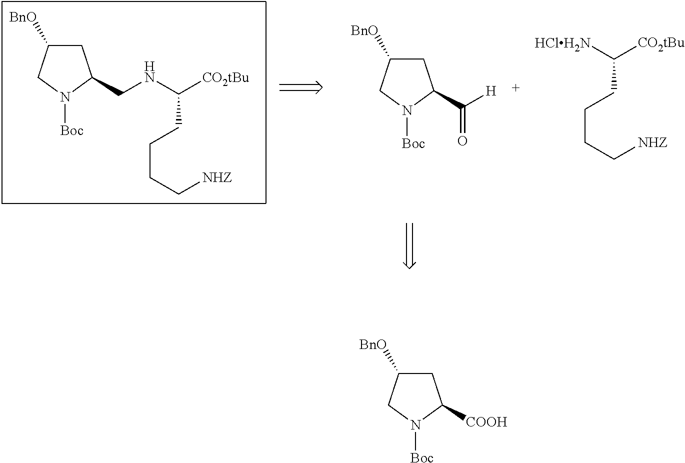

The protected lysine used in this reaction is commercial (Novabiochem ref. 04-12-5122). The aldehyde is synthesized by reduction of the Weinreb amide derived from commercial doubly protected trans-4-hydroxy-L-proline

(Novabiochem ref. 04-10-0020) according to the procedure of Fehrentz and Castro (Fehrentz J.-A., Castro B. Synthesis, 1983, 676-678, [29]).

### 4.1) Synthesis of N^{α}-Boc-trans-4-Hyp(Bn)-N(OMe)Me

The procedure of Fehrentz and Castro ([29]) is applied for the synthesis of the Weinreb amide 81 using the commercial N^{α}-Boc-trans-4-Hyp(Bn)-OH (Novabiochem ref. 04-10-0020) with a yield of 66%. The BOP (benzotriazol-1-yloxytris(dimethylamino)phosphonium hexafluorophosphate) used as coupling agent is replaced with TBTU (2-(1-hydroxybenzotriazol-1-yl)-1,1,3,3-tetrahydrouronium tetrafluoroborate).

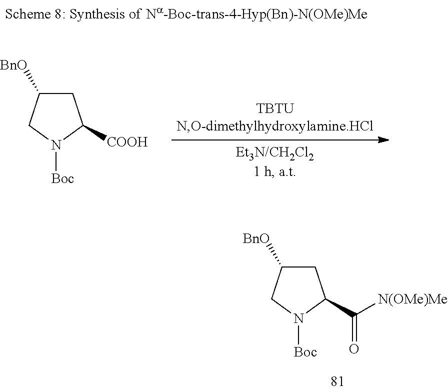

### (4R)-(benzyloxy)-N-(tert-butyloxycarbonyl)-N-methoxy-N-methylpyrrolidin-(2S)-amide 81

257 μl of triethylamine (1.83 mmol; 1 eq) followed by 586 mg of TBTU (1.83 mmol; 1 eq) are added to a solution of commercial N^{α}-Boc-trans-4-Hyp(Bn)-OH. After stirring at ambient temperature for 5 minutes, 214 mg of N,O-dimethylhydroxylamine hydrochloride (2.19 mmol; 1.2 eq) are added along with a further 309 μl of triethylamine (2.2 mmol; 1.2 eq). The reaction is stirred at ambient temperature for 24 hours. 10 ml of CH₂Cl₂ are added and the reaction is washed three times with each of the following solutions: a 10% HCl solution, a saturated solution of NaHCO₃ and a saturated solution of NaCl. The organic phase is dried over anhydrous Na₂SO₄, filtered and concentrated. The more or less colorless oily residue obtained is purified by silica gel column chromatography (EtOAc/PE 50:50). The product (1.063 mmol; 387 mg) is obtained in the form of a yellow oil.

IR (NaCl; film): 3064 and 3031 (v_{φCH}); 2975 and 2935 (v_{CH}); 1736, 1695 and 1683 (v_{C=O}); 1455 (v_{φC=C}); 1403 and 1367 (δ_{CH3} t-Bu); 1250 (v_{C-C-O asym}); 1164 (v_{O-C-C asym}).

¹H NMR (CDCl₃; 300 MHz): 1.43 and 1.47 (9H, 2s, C(CH₃)₃ Boc 1st and 2nd conf.); 2.04 (1H, m, CH_{β}); 2.38 (1H, m, CH_{β}); 3.21 (3H, s, CH₃N); 3.56-3.80 (5H, m+2s, CH₂N and CH₃O 1st and 2nd conf.); 4.19 and 4.26 (1H, 2m, CH_{α} 1st and 2nd conf.); 4.53 (2H, m, CH₂Ph); 4.78 and 4.86 (1H, 2t, J=7.5 Hz and J=7.3 Hz, CHOBn 1st and 2nd conf.); 7.34 (5H, m, Ph).

### 4.2) Synthesis of N^{α}-Boc-trans-4-Hyp(Bn)-al

The Weinreb amide 81 is reduced in the presence of lithium aluminum hydride in ethyl ether for 30 minutes at 0° C. according to the procedure of Fehrentz and Castro ([29]). The yield of the resulting aldehyde 82 is 69%.

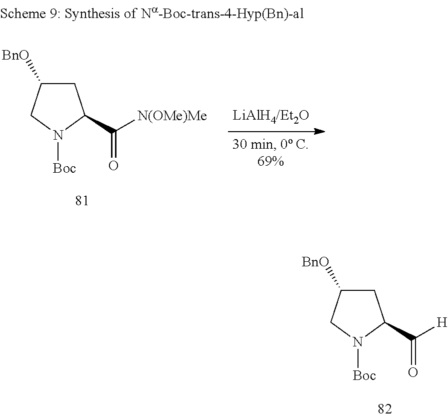

### (4R)-(benzyloxy)-N-(tert-butyloxycarbonyl)pyrrolidin-(2S)-al 82

A solution of N^{α}-Boc-trans-4-Hyp(Bn)-N(OMe)Me 81 (1 mmol; 360 mg) in 6 ml of anhydrous Et₂O is added dropwise to 50 mg of lithium aluminum hydride (1.25 mmol; 1.25 eq) cooled in an ice bath. The mixture is left to stir at 0° C. for 30 minutes and then hydrolyzed with 5 ml of a 0.3 M solution of KHSO₄. The organic phase is separated and the aqueous phase is extracted three times with Et₂O. The ethereal phases are combined and then washed three times with each of the following solutions: a 10% HCl solution, a saturated solution of NaHCO₃ and a saturated solution of NaCl. The final organic phase is dried over anhydrous Na₂SO₄, filtered and concentrated. The oily yellow residue obtained is purified by silica gel column chromatography (EtOAc/PE 40:60). The product (0.688 mmol; 210 mg) is obtained in the form of a yellowish oil.

IR (NaCl; film): 3065 and 3032 (v_{φCH}); 2978 and 2931 (v_{CH}); 2715 (v_{CHO}); 1738, 1704 and 1694 (v_{C=O}); 1455 (v_{φC=C}); 1398 and 1367 (δ_{CH3} t-Bu); 1256 (v_{C-C-O asym}); 1163 (v_{O-C-C asym}).

¹H NMR (CDCl₃; 300 MHz): 1.43 and 1.49 (9H, 2s, C(CH₃)₃ Boc 1st and 2nd conf.); 1.98 (1H, m, CH_{β}); 2.28 (1H, m, CH_{β}); 3.55 and 3.81 (2H, 2m, CH₂N 1st and 2nd conf.); 4.15 (1H, m, CH_{α}); 4.25 and 4.36 (1H, 2m, CHOBn 1st and 2nd conf.); 4.54 (2H, system AB, J=11.6 Hz, CH₂Ph); 7.34 (5H, m, Ph); 9.45 and 9.57 (1H, 2d, J=3.7 Hz and J=2.6 Hz, CHO 1st and 2nd conf.).

### 4.3) Synthesis of N^{α}-Boc-trans-4-Hyp(Bn)-ψ(CH₂—NH)-Lys(Z)-OtBu

The aldehyde 82 is used in a reductive amination reaction with an excess (1.2 equivalents) of commercial protected lysine (Novabiochem ref. 04-12-5122), according to the same procedure as that described in Martinez et al. (Martinez J., Bali J. P., Rodriguez M., Castro B., Magous R., Laur J., Lignon M-F. J. Med. Chem., 1985, 28(12), 1874-1879 [30]). The aminomethyl analog compound No. 6 is obtained after one hour of reaction at ambient temperature with a yield of 52%.

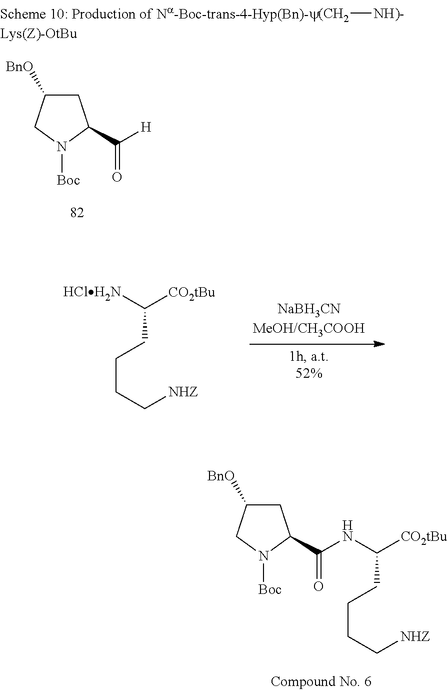

The reduced dipeptide No. 6 is obtained with an overall yield of 24% in three steps using commercial N^{α}-Boc-trans-4-Hyp(Bn)-OH (Novabiochem ref. 04-10-0020).

### N^{α}-Boc-trans-L-4-Hyp(Bn)-ψ(CH₂-NH)-L-Lys(Z)-OtBu 83

The aldehyde 82 (0.68 mmol; 207 mg) is dissolved in 10 ml of MeOH/CH₃COOH (99:1) containing 300 mg of commercial H-Lys(Z)-OtBu.HCl (0.816 mmol; 1.2 eq). 34 mg of sodium cyanoborohydride (0.544 mmol; 0.8 eq) are added in small portions over the course of 45 minutes and with stirring. After stirring for one hour at ambient temperature, the round-bottomed flask is immersed into a bath of ice-cold water, and then 15 ml of a saturated solution of NaHCO₃ are added, followed by 20 ml of EtOAc. The organic phase is separated, washed with 10 ml of water and then dried over anhydrous Na₂SO₄, filtered and concentrated. The oily residue obtained is purified by silica gel column chromatography (EtOAc/PE 40:60). The product (0.350 mmol; 219 mg) is obtained in the form of a yellowish oil.

Elemental analysis: Found C. 66.91%; H, 8.53%; N. 6.77%. C₃₅H₅₁N₃O₇ required C. 67.17%; H, 8.21%; N, 6.71%.

IR (NaCl; film): 3343 (v_{NH}); 3065 and 3033 (v_{φCH}); 2976 and 2933 (v_{CH}); 1728, 1716, 1704, 1694 and 1682 (v_{C=O}) 1455 (v_{φC=C}); 1394 and 1367 (δCH₃ t-Bu), 1247 (v_{C-C-O asym}), 1162 (v_{O-C-C asym}).

¹H NMR (CDCl₃; 300 MHz): 1.46 (9H, 2s, C(CH₃)₃Boc); 1.35-1.68 (6H, m, 3CH₂ Lys); 2.11 (2H, m, CH_{2β}); 2.65 (2H, m, CH₂NH); 3.19 (2H, q, J=6.4 Hz, CH₂NHZ); 3.45 (2H, m, CH₂N); 3.91-4.14 (2H, m, 2CH_{α}); 4.49 (2H, system AB, J=13.3 Hz, CH₂Ph Hyp); 4.80 (1H, M, CHOBn); 5.10 (2H, s, CH₂Ph Z); 7.31-7.38 (10H, m, 2Ph).

¹³C NMR (CDCl₃; 75 MHz): 23.45 (CH₂ Lys); 28.53 and 28.87 (6CH₃ Boc and t-Bu); 30.07 and 33.65 (2CH₂ Lys); 41.27 (CH_{2β3}); 51.13, 51.31 and 51.96 (3CH₂N); 56.65 (CH_{α}); 62.66 (CH_{α}); 66.86 (CH₂Ph Hyp); 71.25 (CH₂Ph Z); 76.60 (CH—O Hyp); 79.96 (C—O t-Bu); 81.39 (C—O t-Bu); 128.06, 128.51, 128.82 and 128.91 (10CH 2Ph); 137.03 (Cq Ph); 138.52 (Cq Ph); 155.37 and 156.78 (2CO Boc and Z); 175.19 (CO ester).

MS (ESI; positive mode): 626.2 [MH]⁺; 570.2 [MH]⁺-isobutene; 514.1 [MH]⁺-2isobutene; 470.3 [MH]⁺-2isobutene—CO₂.

HR MS (ESI; positive mode): 626.38092 [MH]⁺ (calc. 626.3805).

### Example 2

### Biological Activity of Compounds Nos 2, 3, 4, 5 and 6

### 1) Protocols

### 1.1) Cell Cultures

The HEK-293 human fibroblast line (ATCC cell collection CRL-1573) is transfected with either BCRP (Robey R. W. et al. 2003, Br. J. Cancer 89(10) 1971-1978, [32]) or ABCC1, cloned into the pcDNA3.1 expression vector (Invitrogen), or with the empty pcDNA3.1 vector. The NIH3T3 cell line (ATCC CRL-1658) was stably transfected with pHaMDR1/A (Cardarelli et al. 1995 Cancer Res 55 1086-1091, [33]), which is a retroviral vector that expresses the wild-type MDR1 protein. The BHK-21 cell line (ATCC CCL-10) was transfected with MRP1 cloned into the pcDNA3.1 expression vector, or the empty pcDNA3.1 vector. The cells are maintained in culture in DMEM glutamax II (Invitrogen) supplemented with 10% of fetal bovine serum and 1% of penicillin/streptomycin. The data are processed with Sigmaplot V11 (http://www.systat.com/), and the equation for mathematical adjustment of the experimental data for calculating the IC₅₀ values: f=a×(1−exp(−b×x)) (a=slope, b=intercept on the y-axis).

### 1.2) Flow Cytometry

The cells expressing P-gp, BCRP and MRP, and their corresponding controls, are placed in the presence of 10 and 2 μM of mitoxantrone for the first two transporters and of 1 μM of daunorubicin for the 3rd. Substrate accumulation is carried out for 30 minutes at 37° C. in the presence or absence of varied concentrations of the molecules tested, at a final DMSO (dimethyl sulfoxide) concentration of 0.5%. After washing with PBS (phosphate buffered saline), the cells are incubated in medium containing the same concentrations of molecules tested as previously, for 1 h at 37° C. The intracellular fluorescence of mitoxantrone and daunorubicin anticancer agent is measured with the FACscan flow cytometer (Becton Dickinson, Mountain, View, Calif.). The drugs are excited at 488 nm. The emission of mitoxantrone is measured at 650 nm, whereas the emission of daunorubicin is measured at 530 nm. The effectiveness of the molecules tested is expressed by equation 1: % effectiveness=100×(F_{A}−F_{BG})/(F_{C}−F_{BG}), where F_{A} corresponds to the intracellular level of substrate measured in the cells overexpressing ABCG2 (corresponding to the lowest level of fluorescence in the absence of inhibitor), F_{C} corresponds to the fluorescence in the control cells transfected with the empty vector (and therefore to the maximum accumulation of substrate in the cells) and F_{BG} corresponds to the fluorescence measured in the absence of substrate and in the presence of inhibitor.

### 1.3) Cytotoxicity Test

The cells are seeded in a proportion of 10 000 per well in 96-well plates and incubated for 24 hours at 37° C. under 5% CO₂. Varied concentrations of compounds Nos 2, 3, 4, 5 and 6, up to 20 times the IC₅₀, are then added. The cytotoxicity is evaluated colorinnetrically with 3-(4,5-dimethylthiazol-2-yl)-2,5-diphenyltetrazolium bromide (MTT).

### 2) Results

### 2.1) Inhibitory Effect of the Compounds of the Invention on the Drug Efflux Activity of P-gp

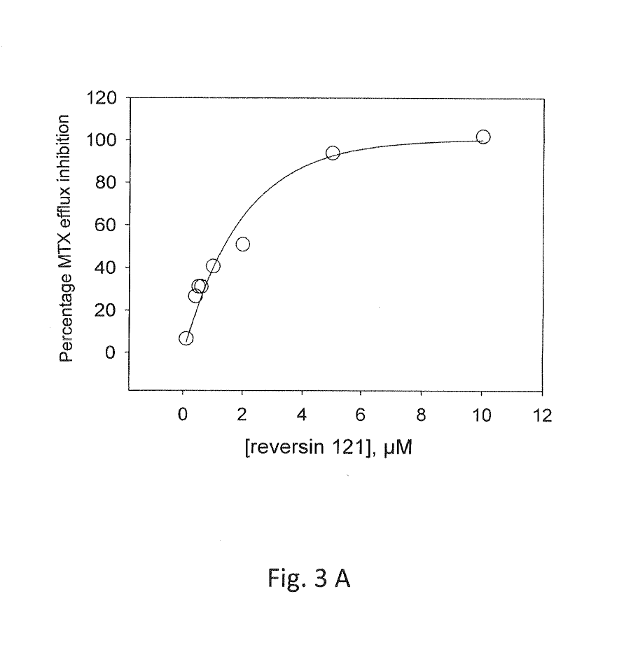

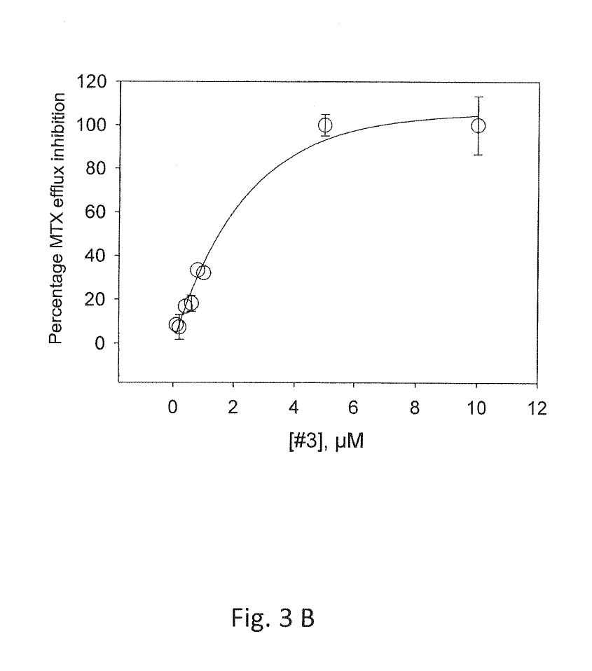

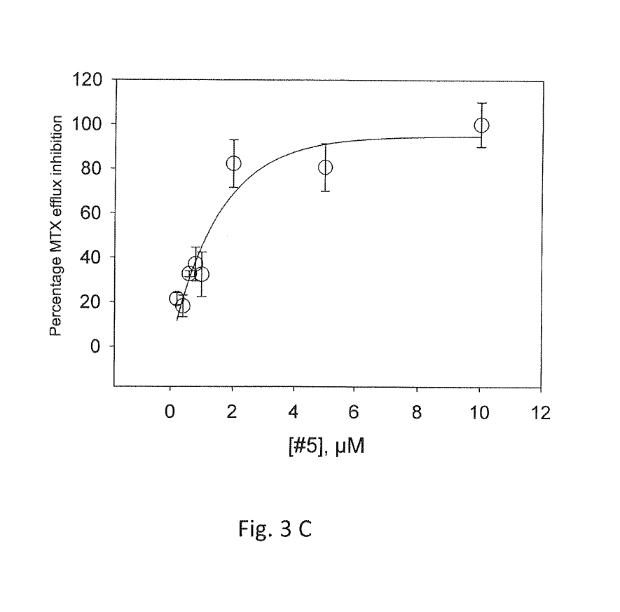

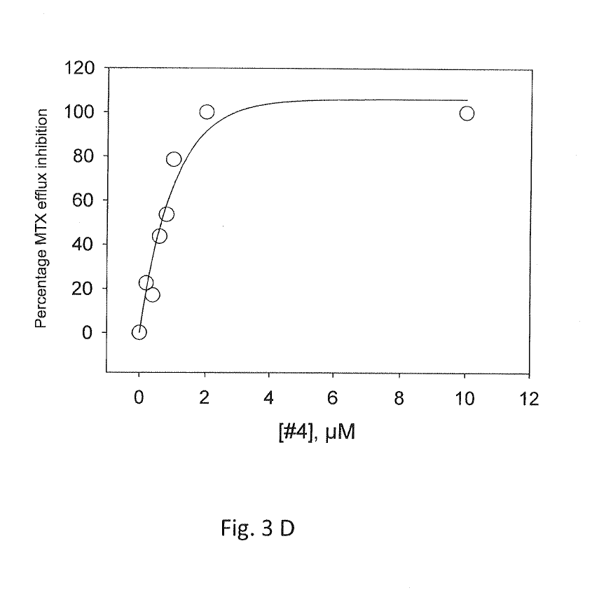

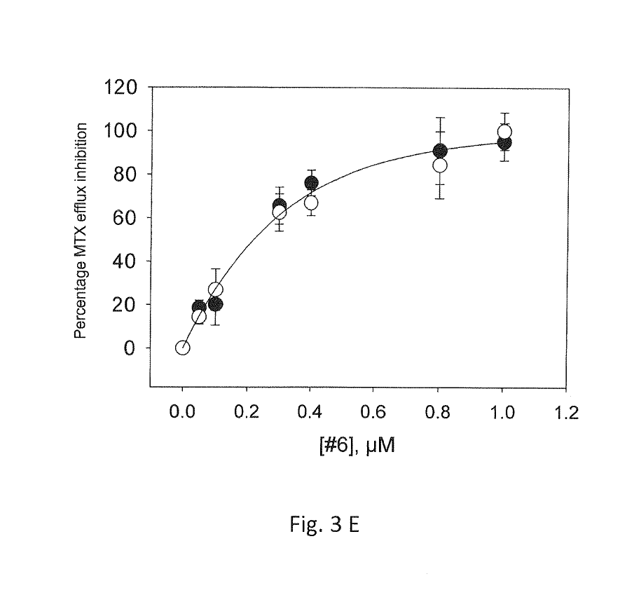

The inhibitory effect of the compounds is quantified by measuring, by flow cytometry, the efflux of an anticancer drug transported by the pump under consideration, i.e. mitoxantrone for P-gp and BCRP and daunorubicin for MRP1. It is compared with that of the reference product, reversin 121. The products are tested at 10 μM, then at 2 μM for those which block at least 80% of the efflux of mitoxantrone, and for some, a more complete concentration range is tested so as to establish the concentration value producing 50% inhibition, IC₅₀, as indicated in FIG. 3. FIG. 3 in fact shows the percentages of inhibition of mitoxantrone efflux as a function of the concentration (μM) of the following compounds: reversin 121, compound No. 3, compound No. 4, compound No. 5, compound No. 6. The corresponding values are indicated in table 2 below.

TABLE 2
Inhibitory effect of the compounds of the invention on MDR ABC
transporters

| | % pump inhibition at 10 μM of compound | | | IC₅₀ |
|---|---|---|---|---|
| # | P-gp | BCRP | MRP1 | P-gp, μM |
| Reversin 121, | 75.8 ± 1.6 | 37.2 ± 3.7 | — | 1.41 ± 0.34 |
| No. 1 | | | | |
| No. 2 | 68.0 ± 10.0 | 10.0 ± 7.5 | — | — |
| No. 3 | 105.7 ± 13.4 | 11.2 ± 4.6 | — | 1.68 ± 0.28 |
| No. 4 | 99.7 ± 11.0 | 16.6 ± 3.4 | <10 | 0.73 ± 0.20 |
| No. 5 | 82.2 ± 10.8 | 18.8 ± 10.0 | — | 1.13 ± 0.34 |
| No. 6 | 106.9 ± 23.0 | 42.3 ± 0.8 | <10 | 0.22 ± 0.03 |

“—” means non measurable

All the compounds are active, and exhibit in particular an activity compatible with their use for reducing the anticancer-agent-efflux activity of P-gp.

Most of the compounds are more active than reversin 121 at a concentration of 10 μM.

The IC₅₀ for P-gp of compound No. 3 is better than that of reversin, and is of the same order of magnitude as the latter. Its BCRP- and MRP1-inhibiting capacity is low or zero, with 11.2% for BCRP and non measurable for MRP1. The IC₅₀ of compound No. 4 is twice as good as that of reversin 121, still with a low or zero BCRP- and MRP1-inhibiting capacity. The IC₅₀ of compound No. 6 is seven times better than that of reversin 121.

### 2.2) Selectivity

Table 2 shows that the compounds are specific for P-gp, and barely or not at all active on BCRP and MRP1, which makes it possible to achieve the objective of having, in a medical context, products which are less toxic, thus preserving the physiological role of the transporters not involved in resistance to a given chemotherapy.

### 2.3) Chemosensitization

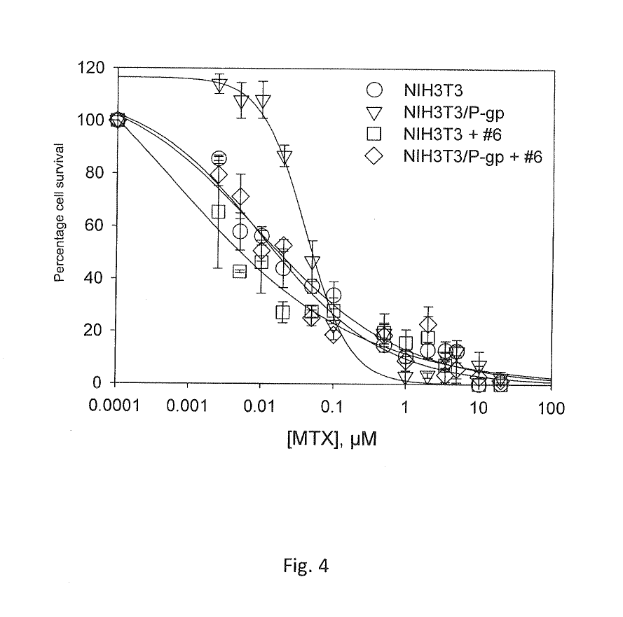

The chemosensitization is illustrated herein in FIG. 4, representing the percentage cell survival of NIH3T3 cells expressing (triangles, diamonds) or not expressing (circles, squares) P-gp, as a function of the concentration (μM) of mitoxantrone administered and of the presence (1 μM, squares, diamonds) or absence (circles, triangles) of compound No. 6.

A similar experiment was carried out with compound No. 4.

In the absence of compound No. 4 or 6, the cells expressing P-gp are ten times more resistant than those not expressing it (triangles compared with circles). The addition of a concentration equivalent to five times the IC₅₀, i.e. 4 μM for compound No. 4 and 1 μM for compound No. 6, completely restores the sensitivity of the cells overexpressing P-gp to the anticancer agent used, mitoxantrone.

### 2.4) Cytotoxicity

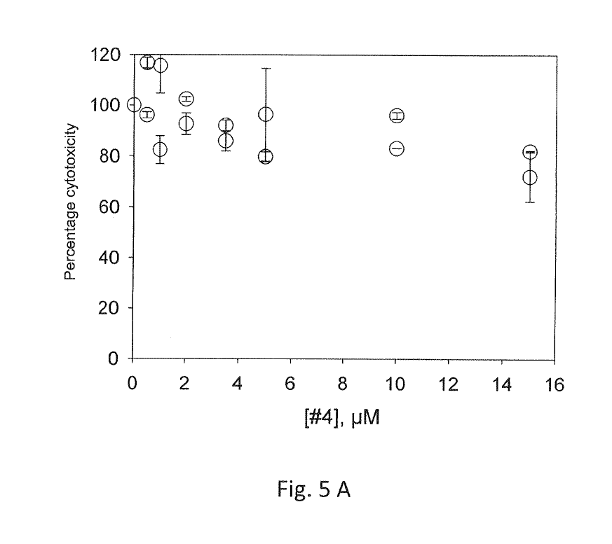

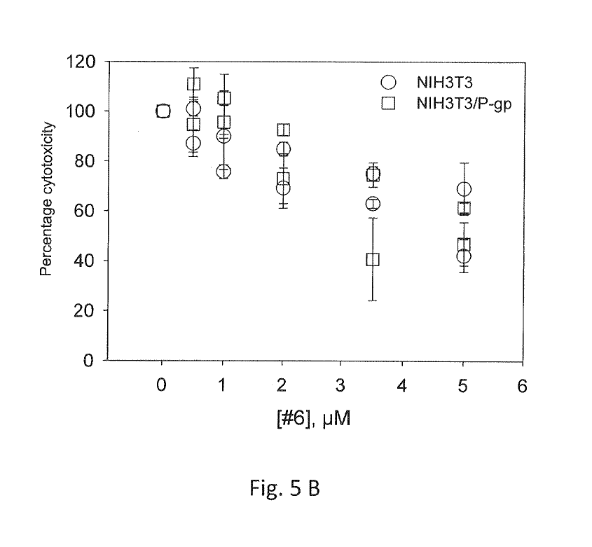

Increasing concentrations of compound No. 4 or No. 6 are applied to NIH3T3 cells expressing or not expressing P-gp. The cell survival is measured after 72 h and is expressed as a percentage. The cytotoxicity is shown in FIG. 5. At 10 times the half-maximum-effect concentration, these two compounds are not toxic. Above this, the toxicity of compound No. 4 remains negligible, while that of compound No. 6 remains limited.

This lack of cytotoxicity is therefore compatible with the administration of these compounds in a mammal.

### 2.5) Mode of Inhibition

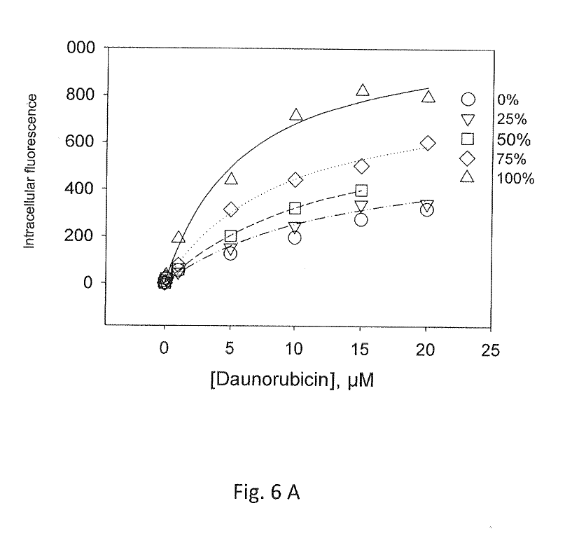

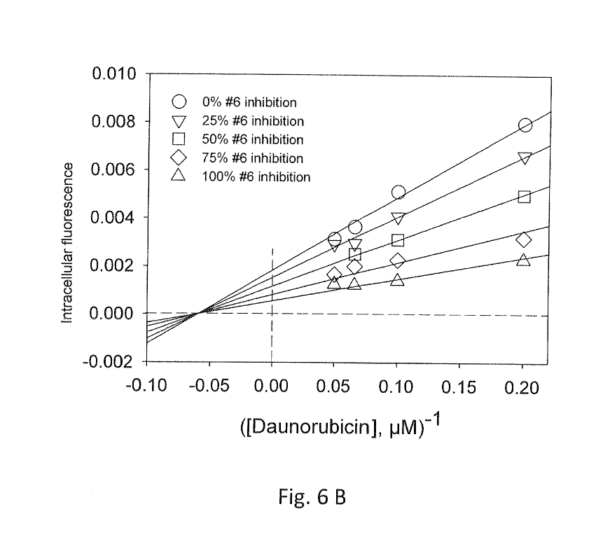

FIG. 6 clearly shows that the mechanism of inhibition by compound No. 6 is of the noncompetitive type, particularly obvious in the Lineweaver & Burke double reciprocal representation on the right-hand panel. The intracellular concentration of fluorescent daunorubicin is measured at increasing daunorubicin concentrations, in the presence or absence of fixed concentrations of 0, 0.12 μM, 0.22 μm, 0.44 μM and 10 μM of compound No. 6 corresponding to 0%, 25%, 50%, 75% and 100% efflux inhibition effectiveness according to FIG. 3. The double reciprocal representation (right-hand panel) shows that the lines out the x-axis at a single point, typical of a noncompetitive type of inhibition.

### LIST OF REFERENCES

1. Gottesman M. M., Ling V. FEBS Lett., 2006, 580(4), 998-1009

2. Juliano, R. L. & Ling, V. A surface glycoprotein modulating drug permeability in Chinese hamster ovary cell mutants. Biochim Biophys Acta 455, 152-162, doi:0005-2736(76)90160-7 [pii] (1976)

3. Cole, S. P. et al. Overexpression of a transporter gene in a multidrug-resistant human lung cancer cell line. Science 258, 1650-1654 (1992)

4. Doyle L. A., Yang W., Abruzzo L. V., Krogmann T., Gao Y., Rishi A. K., Ross D. D. Proc. Natl. Acad. Sci. USA, 1998, 95(26), 15665-15670

5. Ross, D. D. et al. Atypical multidrug resistance: breast cancer resistance protein messenger RNA expression in mitoxantrone-selected cell lines. J Natl Cancer Inst 91, 429-433 (1999)

6. J. Cell Sci., 2000, 113(Pt 11), 2011-2021

7. Aller, S. G. et al. Structure of P-Glycoprotein Reveals a Molecular Basis for Poly-Specific Drug Binding. Science 323, 1718-1722, doi:10.1126/science.1168750 (2009)

8. Dawson, R. J. & Locher, K. P. Structure of a bacterial multidrug ABC transporter. Nature 443, 180-185 (2006)

9. Ward, A., Reyes, C. L., Yu, J., Roth, C. B. & Chang, G. Flexibility in the ABC transporter MsbA: Alternating access with a twist. Proceedings of the National Academy of Sciences 104, 19005-19010, doi:10.1073/pnas.0709388104 (2007)

10. Ueda, K., Cardarelli, C., Gottesman, M. M. & Pastan, I. Expression of a Full-Length cDNA for the Human “MDR1” Gene Confers Resistance to Colchicine, Doxorubicin, and Vinblastine. Proceedings of the National Academy of Sciences 84, 3004-3008, doi:10.1073/pnas.84.9.3004 (1987)

11. Litman, T. et al. The multidrug-resistant phenotype associated with overexpression of the new ABC half-transporter, MXR (ABCG2). J Cell Sci 113 (Pt 11), 2011-2021 (2000)

12. Benderra, Z. et al. Breast Cancer Resistance Protein and P-Glycoprotein in 149 Adult Acute Myeloid Leukemias. Clin Cancer Res 10, 7896-7902, doi:10.1158/1078-0432.ccr-04-0795 (2004)

13. Juliano, R. L. & Ling, V. A surface glycoprotein modulating drug permeability in Chinese hamster ovary cell mutants. Biochim Biophys Acta 455, 152-162, doi:0005-2736(76)90160-7 [pii] (1976)

14. W. Clark Still, Michael Kahn, and Abhijit Mitra J. Org. Chem. Rapid Chromatographic Technique for Preparative Separations with Moderate Resolution, J. Org. Chem., 1978, 43(14), 2923-2925

15. Kim, R. B. Drug transporters in HIV Therapy. Top HIV Med 11, 136-139 (2003)

16. Allikments R., Schriml L. M., Hutchinson A., Romano-Spica V., Dean M. Cancer Res., 1998, 58(23), 5337-5339

17. Cooray H. C., Janvilisri T., Van Veen H. W., Hladky S. B., Barrand M. A. Biochem. Biophys. Res. Commun., 2004, 317(1), 269-275

18. Jonker J. W., Smit J. W., Brinkhuis R. F., Maliepaard M., Beijnen J. H., Schellens J. H. J. Natl. Cancer Inst., 2000, 92, 1651-1656

19. Modok, S., Mellor, H. R. & Callaghan, R. Modulation of multidrug resistance efflux pump activity to overcome chemoresistance in cancer. Current Opinion in Pharmacology 6, 350-354 (2006)

20. Current Opinion in Pharmacology, 2006, 6, 350-354

21. Sarkadi B., Seprodi J., Csuka O., Magocsi M., Mezõ I., Palyi I., Teplán I., Vadász Z., Vincze, B. U.S. Pat. No. 6,297,216 B1, 2 Oct. 2001

22. Seprödi J., Mezõ I., Vadász Zs., Szabó K., Sarkadi B., Teplán I. in peptides 1996, Proceedings of the Twenty-Fourth European Peptide Symposium; Sep. 8-13, 1996, Edinburgh, Scotland. Robert Ramage and Roger Epton (Eds), 1998, pp. 801-802

23. Sharom F. J., Yu X., Lu P., Liu R., Chu J. W. K., Szabó K., Muller M., Hose C. D., Monks A., Varadi A., Seprödi J., Sarkadi B. Biochem. Pharmacol., 1999, 58(4), 571-586

24. Thesis, Synthesis and biological study of di- and tripeptidic analogs of reversins that may modulate the activity of two transport proteins, P-glycoprotein and BCRP (2007)

25. Lai, M. Y. H. et al. Synthesis and pharmacological evaluation of glycine-modified analogs of the neuroprotective agent glycyl-l-prolyl-l-glutamic acid (GPE). Bioorganic & Medicinal Chemistry 13, 533-548 (2005)

26. Martinez, J. et al. Synthesis and biological activities of some pseudo-peptide analogs of tetragastrin: the importance of the peptide backbone. Journal of Medicinal Chemistry 28, 1874-1879 (1985)

27. Palyi, I. et al. Compounds for reversing drug resistance. Hungary patent (2001)

28. Seko T., Kato M., Kohno H., Ono S., Hashimura K., Takimizu H., Nakai K., Maegawa H., Katsube N., Toda M. Bioorg. Med. Chem., 2003, 11(8), 1901-1913

29. Fehrentz J.-A., Castro B. Synthesis, 1983, 676-678

30. Martinez J., Bali J. P., Rodriguez M., Castro B., Magous R., Laur J., Lignon M-F. J. Med. Chem., 1985, 28(12), 1874-1879

31. Carrasco M. R., Brown R. T. J. Org. Chem., 2003, 68(23), 8853-8858

32. Robey R. W. et al. 2003, Br. J. Cancer 89(10) 1971-1978

33. Cardarelli et al. 1995 Cancer Res 55 1086-1091

34. Vogel's textbook of practical organic chemistry, Vth edition, 1989, published by Longman Scientific & Technical

1-15. (canceled)

16. A pharmaceutical composition comprising a compound of formula (I) or a salt thereof and a pharmaceutically acceptable carrier: 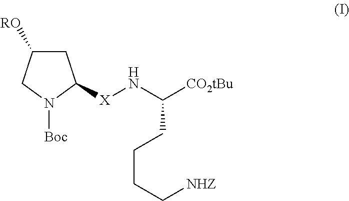
wherein:
R represents -Bn, —COBn, —COcHex or —CH₂cHex,
X represents —CH₂ or —CO,
Z represents a benzyloxycarbonyl group.

17. The pharmaceutical composition according to claim 16, wherein:
R represents -Bn and X represents CH₂, or
R represents —COBn and X represents CO, or
R represents —CocHex and X represents CO, or
R represents -Bn and X represents CO, or
R represents —CH₂cHex and X represents CO.

18. The pharmaceutical composition according to claim 16, wherein the concentration of said compound of structure (I) or a salt thereof is between 0.001 and 500 μM.

19. The pharmaceutical composition according to claim 16, further comprising a chemotherapeutic agent.

20. The pharmaceutical composition according to claim 19, wherein said chemotherapeutic agent is an anticancer agent or an anti-infective agent.

21. The pharmaceutical composition according to claim 16, wherein said compound of formula (I) or a salt thereof is intended for treating a cancer or an infection.

22. The pharmaceutical composition according to claim 16, wherein said compound of structure (I) or a salt thereof is an adjuvant to a chemotherapy treatment of a cancer or of an infection.

23. The pharmaceutical composition according to claim 16, wherein said compound of structure (I) or a salt thereof has a half-maximum inhibition concentration (IC₅₀) of about 0.22 μM.

24. A method for treating a cancer or an infection in a mammal in need thereof, comprising administrating to said mammal an effective amount of a compound of formula (I) or a salt thereof: 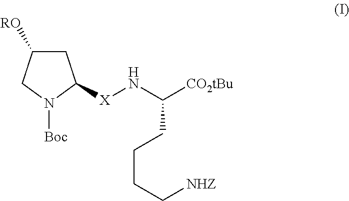
wherein:
R represents -Bn, —COBn, —COcHex or —CH2cHex,
X represents —CH2 or —CO,
Z represents a benzyloxycarbonyl group.

25. The method for treating a cancer or an infection according to claim 24, wherein:
R represents -Bn and X represents CH2, or
R represents —COBn and X represents CO, or
R represents —CocHex and X represents CO, or
R represents -Bn and X represents CO, or
R represents —CH2cHex and X represents CO.

26. The method for treating a cancer or an infection according to claim 24, wherein said compound of formula (I) or a salt thereof is administered in combination with a chemotherapeutic agent.

27. The method for treating a cancer or an infection according to claim 26, wherein said chemotherapeutic agent is an anticancer agent or an anti-infective agent.

28. A method for boosting a chemotherapy treatment of a cancer or of an infection in a mammal in need thereof, comprising administrating to said mammal an effective amount of a compound of formula (I) or a salt thereof in combination with a chemotherapeutic agent intended for treating a cancer or an infection: 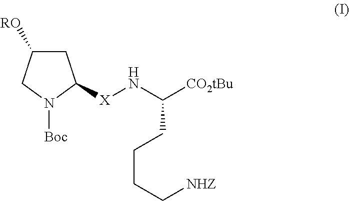
wherein:
R represents -Bn, —COBn, —COcHex or —CH2cHex,
X represents —CH2 or —CO,
Z represents a benzyloxycarbonyl group.

29. The method for boosting a chemotherapy treatment of a cancer or of an infection according to claim 28, wherein:
R represents -Bn and X represents CH2, or
R represents —COBn and X represents CO, or
R represents —CocHex and X represents CO, or
R represents -Bn and X represents CO, or
R represents —CH2cHex and X represents CO.

30. The method for boosting a chemotherapy treatment of a cancer or of an infection according to claim 28, wherein said compound of formula (I) or a salt thereof is intended for reducing the resistance to said chemotherapeutic agent.

31. The method for boosting a chemotherapy treatment of a cancer or of an infection according to claim 28, wherein said chemotherapeutic agent is an anticancer agent or an anti-infective agent.

32. A method for reducing the activity of the ABCB1 efflux protein in a mammal or in vitro, comprising administrating respectively to said mammal or in vitro an effective amount of a compound of formula (I) or a salt thereof: 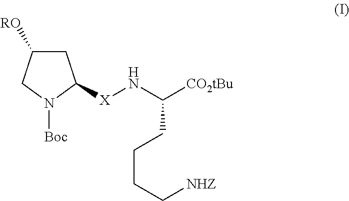
wherein:
R represents -Bn, —COBn, —COcHex or —CH2cHex,
X represents —CH2 or —CO,
Z represents a benzyloxycarbonyl group.

33. The method for reducing the activity of the ABCB1 efflux protein according to claim 32, wherein:
R represents -Bn and X represents CH2, or
R represents —COBn and X represents CO, or
R represents —CocHex and X represents CO, or
R represents -Bn and X represents CO, or
R represents —CH2cHex and X represents CO.
